(19) United States
(12) Patent Application Publication
(10) Pub. No.: US 2025/0179040 A1
(43) Pub. Date: Jun. 5, 2025

(54) HISTAMINE H3 RECEPTOR ANTAGONISTS AND THEIR PHARMACEUTICAL USES

(71) Applicant: HANGZHOU BIO-SINCERITY PHARMA-TECH CO., LTD., Hangzhou (CN)

(72) Inventors: Enguang Feng, Hangzhou (CN); Jinjing Qin, Hangzhou (CN); Lifeng Jin, Hangzhou (CN); Zewu Jin, Hangzhou (CN); Yuanyuan Li, Hangzhou (CN); Xiaohong Xiong, Hangzhou (CN); Shaowei Lei, Hangzhou (CN); Ximing Shen, Hangzhou (CN); Jinfang Lou, Hangzhou (CN)

(21) Appl. No.: 18/843,289

(22) PCT Filed: Mar. 2, 2023
(86) PCT No.: PCT/IB2023/000170

§ 371 Date: Sep. 2, 2024

(30) Foreign Application Priority Data
Mar. 2, 2022 (CN) 202210200094.1

(51) Int. Cl. C07D 295/15; A61K 31/451; A61K 31/454; A61K 31/4545; A61K 31/496; A61K 31/5377; A61K 31/541; A61K 31/551; A61P 17/04; A61P 25/04; A61P 29/00; C07D 207/06; C07D 211/22; C07D 211/38; C07D 211/44; C07D 295/096; C07D 295/13

(52) U.S. Cl. CPC C07D 295/15; A61K 31/451; A61K 31/454; A61K 31/4545; A61K 31/496; A61K 31/5377; A61K 31/541; A61K 31/551; A61P 17/04; A61P 25/04; A61P 29/00; C07D 207/06; C07D 211/22; C07D 211/38; C07D 211/44; C07D 295/096; C07D 295/13

(57) ABSTRACT

The present invention discloses a series of structurally novel histamine H3 receptor antagonists, specifically relating to the compounds as free base, or isomer, or solvate, or pharmaceutically acceptable salt forms; methods of preparing medicaments; compounds composition, and therapeutic uses thereof. The present invention has developed a series of structurally novel compounds based on histamine H3 receptor ligands, and a series of relevant biological tests have been performed on the compounds. The results of the tests all indicate that the compounds have significant H3 receptor antagonistic activity, and can be used as lead compounds for the prevention or treatment H3 receptor-related diseases.

### CROSS-REFERENCE TO RELATED APPLICATIONS

This patent application is the national phase application of PCT Application No. PCT/IB2023/000170, filed on Mar. 2, 2023, which claims priority to Chinese patent application No. 202210200094.1, filed on Mar. 2, 2022, the contents of which are incorporated herein by reference in their entirety.

### BACKGROUND OF THE INVENTION

Following the Human Genome Project, the histamine receptor family has expanded to four distinct G protein-coupled receptors (GPCRs): the H1, H2, H3, and H4 receptors (Nature Review Drug Discovery, 2005, 4, 107-120). Histamine H3 receptors are mainly expressed in the central nervous system and less frequently in the peripheral nervous system.

Histamine H3 receptor (H3R) is widely expressed in the brain, mainly in the cerebral cortex, hippocampus, amygdala, striatum and other areas closely related to memory and cognitive abilities. Blocking these receptors with selective antagonists/inverse agonists can increase the release of neurotransmitters such as acetylcholine, dopamine or 5-hydroxytryptamine, thereby regulating a variety of neuropathic behaviors such as learning and memory, wakefulness and sleep (British Journal of Pharmacology, 2008, 154(6), 1166-1181).

Literatures suggests that ligands of histamine H3 receptor can be used to treat cognitive impairment (British Journal of Pharmacology, 2008, 154(6), 1166-1181), dementia (Drug News Perspective, 2010, 23(2), 99-103), attention deficit hyperactivity disorder, obesity (Indian Journal of Pharmacology, 2001, (33), 17-28), schizophrenia (Biochemical Pharmacology, 2007, 73(8), 1215-1224), and pain (Journal of Pharmacology and Experimental Therapeutics, 2011, 336(1), 30-37).

Presently, histamine H3 receptor ligands compounds are mainly divided into two categories: imidazole ring substituted at the 4 (5) position and non-imidazole ring. Compounds containing imidazole rings have the disadvantages of low blood-brain barrier penetration, interaction with cytochrome P-450 proteins, liver toxicity, and ocular toxicity. Representative patents of non-imidazole ring compounds: \textcircled{1} US 20020177589 A1 discloses new ligands that can be used to regulate histamine H3 receptor. The compounds in this patent can be used to treat Alzheimer's disease and cognitive impairment. The representative compound is ABT-239, which has serious cardiac side effects in clinical trials, resulting in the termination of clinical trials. \textcircled{2} WO 2012114348 A1 discloses novel compounds as ligands of histamine H3 receptor, which can be used to treat various diseases, such as cognitive impairment, dementia, etc. Samelisant (SUVN-G3031), an H3R antagonist/inverse agonist for the treatment of cognitive impairment, is currently under development. The drug's Phase II clinical indication is narcolepsy, and the Phase I clinical trial for cognitive impairment was conducted from 2015 to 2017, with no subsequent progress. \textcircled{3} WO 2014030170 A1 discloses pyridazinone derivatives as histamine H3 inhibitor, as well as their uses, preparation methods and pharmaceutical compositions. The inventive compounds can be used to treat various diseases such as cognitive impairment, sleep/wakefulness disorders, eating disorders, etc., but the clinical trial was terminated due to the unsatisfactory PK/PD data.

In summary, while a number of histamine H3 receptor ligands analogs are disclosed, only Pitolisant (H3R antagonist/inverse agonist) has been approved for marketing in this area of research and development to date, with the marketed indication of narcolepsy. SUVN-G3031 and other investigational drugs in Phase 2 clinical studies are still narcolepsy, the clinical studies in neurological disorders such as cognitive impairment have been declared failures.

Therefore, there is an urgent need to discover a new compound with novel structure, high safety profile, and better drug's efficacy than Pitolisant and SUVN-G3031, which is used to prevent or treat a disease associated with H3 targets. This is still not solved by those skilled in the art.

### SUMMARY OF THE INVENTION

The present invention aims to provide a structurally novel histamine H3 receptor antagonist which can be used to prevent or treat the diseases associated with histamine H3 receptor.

To achieve the above-mentioned purpose of the invention, the technical solution provided by the present invention is as follows:

A histamine H3 receptor antagonist, is a compound of general formula I or a pharmaceutically acceptable salt thereof:

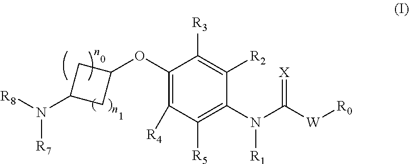

wherein:
W is selected from —(CH₂)ₘ or —NR₆(CH₂)ₘ—;
X is selected from O or S;
n₀ and n₁ are each independently selected from 1, 2 or 3; m is selected from 0, 1, 2, 3, 4, 5 or 6;
R₀ is selected from —NR′R″, —O(CH₂)ₘNR′R″, substituted or unsubstituted 5-8 membered heterocyclyl, substituted or unsubstituted 5-8 membered heteroaryl; wherein the heterocyclyl or heteroaryl is each independently optionally substituted with one or more substituent: C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, C₁₋₆ alkylsulfoxide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-6 membered cycloalkyl, 3-6 membered heterocyclyl, aryl or heteroaryl;
R′ and R″ are each independently selected from hydrogen, C₁₋₆ alkyl, C₁₋₆ alkoxy, halogen, amino, hydroxy, carboxyl, carbonyl, amide, cyano, C₁₋₆ haloalkyl, C₁₋₆ alkylhydroxy, C₁₋₆ alkylamino, C₁₋₆ alkylamide, C₁₋₆ haloalkoxy, C₁₋₆ alkoxyhydroxy, C₁₋₆ alkoxyamino, and C₁₋₆ alkoxyamide, and R′ and R″ are not hydrogen at the same time;
R₁ and R₆ are each independently selected from hydrogen, C₁₋₆ alkyl, —C(O)-alkyl or —S(O)₂-alkyl;
R₂, R₃, R₄, and R₅ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, and C₁₋₆ alkylsulfoxide;
or, R₂, R₃ and the two carbon atoms on the benzene ring to which R₂, R₃ are connected respectively, form a substituted or unsubstituted benzobicyclic structure, wherein the benzobicyclic structure may be a benzoheterocyclic ring, but is not limited thereto;
or, R₄, R₅ and the two carbon atoms on the benzene ring to which R₄, R₅ are connected respectively, form a substituted or unsubstituted benzobicyclic structure; wherein the benzobicyclic structure may be a benzoheterocyclic ring, but is not limited thereto;
R₇ and R₈ are each independently selected from hydrogen, halogen, C₁₋₆ alkyl, C₂₋₆ alkenyl, C₂₋₆ alkynyl, amino, cyano, amide, sulfone, sulfoxide, C₁₋₆ haloalkyl, C₁₋₆ alkylhydroxy, C₁₋₆ alkylamino, C₁₋₆alkylamide, C₁₋₆ haloalkoxy, C₁₋₆alkoxyhydroxy, C₁₋₆alkoxyamino, C₁₋₆alkoxyamide, C₁₋₆alkoxysulfone and C₁₋₆alkoxysulfoxide;
or, R₇, R₈ and the nitrogen atom to which they are connected form a ring, and the substituents on the ring are one or more selected from the following groups: hydrogen, halogen, C₁₋₆ alkyl, C₁₋₆ alkoxy, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-8 membered heterocyclyl, or 3-8 membered heteroaryl;
the heterocyclyl, heteroaryl and benzoheterocyclic groups each contain at least one heteroatom, and the heteroatom is selected from N, O or S.

Preferably, the compound of general formula II or a pharmaceutically acceptable salt thereof:

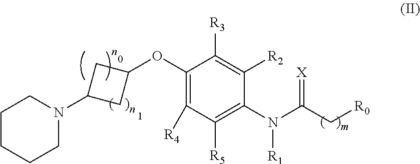

wherein:
X is selected from O or S;
n₀ and n₁ are each independently selected from 1, 2 or 3; m is selected from 0, 1, 2, 3, 4, 5 or 6;
R₀ is selected from —NR′R″, —O(CH₂)ₘNR′R″, substituted or unsubstituted 5-8 membered heterocyclyl, substituted or unsubstituted 5-8 membered heteroaryl; wherein the heterocyclyl or heteroaryl is each independently optionally substituted with one or more substituent: C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, C₁₋₆ alkylsulfoxide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-6 membered cycloalkyl, 3-6 membered heterocyclyl, aryl or heteroaryl;
R′ and R″ are each independently selected from hydrogen, C₁₋₆ alkyl, C₁₋₆ alkoxy, halogen, amino, hydroxy, carboxyl, carbonyl, amide, cyano, C₁₋₆ haloalkyl, C₁₋₆ alkylhydroxy, C₁₋₆ alkylamino, C₁₋₆ alkylamide, C₁₋₆ haloalkoxy, C₁₋₆ alkoxyhydroxy, C₁₋₆ alkoxyamino, and C₁₋₆ alkoxyamide, and R′ and R″ are not hydrogen at the same time;
R₁ is selected from hydrogen, C₁₋₆ alkyl, —C(O)-alkyl or —S(O)₂-alkyl;
R₂, R₃, R₄, and R₅ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, and C₁₋₆ alkylsulfoxide;
or, R₂, R₃ and the two carbon atoms on the benzene ring to which R₂, R₃ are connected respectively, form a substituted or unsubstituted benzobicyclic structure, wherein the benzobicyclic structure may be a benzoheterocyclic ring, but is not limited thereto;
or, R₄, R₅ and the two carbon atoms on the benzene ring to which R₄, R₅ are connected respectively, form a substituted or unsubstituted benzobicyclic structure; wherein the benzobicyclic structure may be a benzoheterocyclic ring, but is not limited thereto;
the heterocyclyl, heteroaryl and benzoheterocyclic groups each contain at least one heteroatom, and the heteroatom is selected from N, O or S.

Preferably, the compound of general formula II-1 or a pharmaceutically acceptable salt thereof:

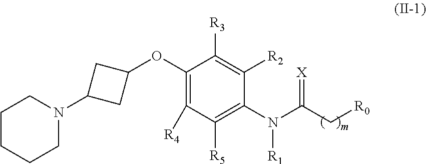

wherein:
X is selected from O or S;
m is selected from 1 or 2;
R₀ is selected from one of substituted or unsubstituted 5-8 membered heterocyclyl and substituted or unsubstituted 5-8 membered heteroaryl; wherein the heterocyclyl or heteroaryl is each independently optionally substituted with one or more substituent: C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆alkylhydroxyl, C₁₋₆ alkylamino, C₁₋₆ alkylamide, C₁₋₆ alkylsulfone, C₁₋₆ alkylsulfoxide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-6 membered cycloalkyl, 3-6 membered heterocyclyl, aryl and heteroaryl;
R₁ is selected from hydrogen, C₁₋₆ alkyl, —C(O)-alkyl or —S(O)₂-alkyl;
R₂, R₃, R₄, and R₅ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, and C₁₋₆ alkylsulfoxide;
the heterocyclyl, heteroaryl and benzoheterocyclic groups each contain at least one heteroatom, and the heteroatom is selected from N, O or S.

Preferably, the compound of general formula II-2 or a pharmaceutically acceptable salt thereof:

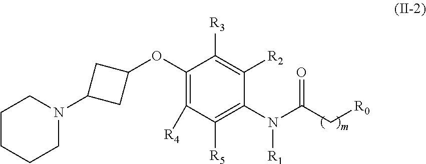

wherein:
m is selected from 1 or 2;
R₀ is selected from —NR′R″ or —O(CH₂)ₘNR′R″;
R′ and R″ are each independently selected from hydrogen, C₁₋₆ alkyl, C₁₋₆ alkoxy, halogen, amino, hydroxy, carboxyl, carbonyl, amide, cyano, C₁₋₆ haloalkyl, C₁₋₆alkylhydroxy, C₁₋₆ alkylamino, C₁₋₆alkylamide, C₁₋₆ haloalkoxy, C₁₋₆alkoxyhydroxy, C₁₋₆ alkoxyamino, and C₁₋₆ alkoxyamide, and R′ and R″ are not hydrogen at the same time;
R₁ is selected from hydrogen, C₁₋₆ alkyl, —C(O)-alkyl or —S(O)₂-alkyl;
R₂, R₃, R₄, and R₅ are each independently one or more selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, and C₁₋₆ alkylsulfoxide.

Preferably, the compound of general formula III or a pharmaceutically acceptable salt thereof:

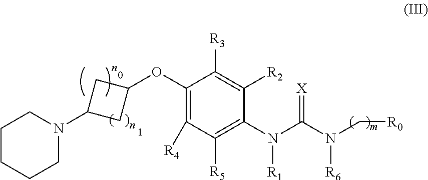

wherein:
X is selected from O or S;
n₀ and n₁ are each independently selected from 1, 2 or 3; m is selected from 0, 1, 2, 3, 4, 5 or 6;
R₀ is selected from one of substituted or unsubstituted 5-8 membered heterocyclyl and substituted or unsubstituted 5-8 membered heteroaryl; wherein the heterocyclyl or heteroaryl is each independently optionally substituted with one or more substituent: C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆ alkylhydroxyl, C₁₋₆ alkylamino, C₁₋₆alkylamide, C₁₋₆alkylsulfone, C₁₋₆alkylsulfoxide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-6 membered cycloalkyl, 3-6 membered heterocyclyl, aryl and heteroaryl;
R₁ and R₆ are each independently selected from hydrogen, C₁₋₆ alkyl, —C(O)-alkyl or —S(O)₂-alkyl;
R₂, R₃, R₄, R₅ and R₆ are each independently one or more selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, and C₁₋₆ alkylsulfoxide;
or, R₂, R₃ and two carbon atoms on the benzene ring, to which R₂, R₃ are connected respectively, form a substituted or unsubstituted benzobicyclic structure; wherein the benzobicyclic structure may be a benzoheterocyclic ring, but is not limited thereto;
or, R₄, R₅ and two carbon atoms on the benzene ring, to which R₄, R₅ are connected respectively, form a substituted or unsubstituted benzobicyclic structure; wherein the benzobicyclic structure may be a benzoheterocyclic ring, but is not limited thereto;
the heterocyclyl, heteroaryl and benzoheterocyclic groups each contain at least one heteroatom, and the heteroatom is selected from N, O or S.

Preferably, the compound of general formula III-1 or a pharmaceutically acceptable salt thereof:

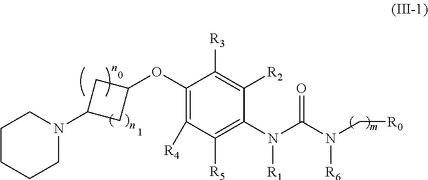

wherein:
n₀ and n₁ are each independently selected from 1, 2 or 3; m is selected from 0, 1, 2, 3, 4, 5 or 6;
R₀ is selected from one of substituted or unsubstituted 5-8 membered heterocyclyl and substituted or unsubstituted 5-8 membered heteroaryl; Wherein the heterocyclyl or heteroaryl is each independently optionally substituted with one or more of the following groups: C₁₋₆ alkyl, C₁₋₆ alkoxy, C₂₋₆ alkenyl, C₂₋₆ alkynyl, halogen, amino, hydroxyl, C₁₋₆ haloalkyl, C₁₋₆ alkylhydroxyl, C₁₋₆ alkylamino, C₁₋₆ haloalkoxy, C₁₋₆ alkoxyhydroxyl, C₁₋₆alkoxyamino, 3-6 membered cycloalkyl;
R₂, R₃, R₄, R₅, and R₆ are each independently one or more selected from hydrogen, halogen, C₁₋₆ alkyl, amino, hydroxy, C₁₋₆ haloalkyl, C₁₋₆alkylhydroxy, and C₁₋₆alkylamino; the heterocyclyl, heteroaryl and benzoheterocyclic groups each contain at least one heteroatom, and the heteroatom is selected from N, O or S.

The present invention also provides a histamine H3 receptor antagonist, include a series of compounds as numbered in BIOS-B-1 to BIOS-B-31, or isomers thereof, or solvates thereof, or pharmaceutically acceptable salts thereof:
BIOS-B-1: 2-morpholino-N-(4-(3-(piperidin-1-yl)cyclobutoxy)phenyl)acetamide;
BIOS-B-2: (R)-2-(2-methylpyrrolidin-1-yl)-N-(4-(3-(piperidin-1-yl)cyclobutoxy)phenyl)acetamide;
BIOS-B-3: 2-(piperidin-1-yl)-N-(4-(3-(piperidin-1-yl)cyclobutoxy)phenyl)acetamide;
BIOS-B-4: 2-(4-methyl-1,4-diazepan-1-yl)-N-(4-(3-(piperidin-1-yl)cyclobutoxy)phenyl)acetamide;
BIOS-B-5: 2-(4,4-difluoropiperidin-1-yl)-N-(4-(3-(piperidin-1-yl)cyclobutoxy)phenyl)acetamide;
BIOS-B-6: 2-(4-hydroxypiperidin-1-yl)-N-(4-(3-(piperidin-1-yl)cyclobutoxy)phenyl)acetamide;
BIOS-B-7: N-(4-(3-(piperidin-1-yl)cyclobutoxy)phenyl)-2-thiomorpholinoacetamide;
BIOS-B-8: 2-(3-(2-hydroxyethyl)piperidin-1-yl)-N-(4-(3-(piperidin-1-yl)cyclobutoxy)phenyl)acetamide;
BIOS-B-9: 2-(piperazin-1-yl)-N-(4-(3-(piperidin-1-yl)cyclobutoxy)phenyl)acetamide;
BIOS-B-10: 2-(4-methylpiperazin-1-yl)-N-(4-(3-(piperidin-1-yl)cyclobutoxy)phenyl)acetamide;
BIOS-B-11: 2-(3-hydroxypiperidin-1-yl)-N-(4-(3-(piperidin-1-yl)cyclobutoxy)phenyl)acetamide;
BIOS-B-12: 2-(4-cyclobutylpiperazin-1-yl)-N-(4-(3-(piperidin-1-yl)cyclobutoxy)phenyl)acetamide;
BIOS-B-13: 1-(2-morpholinoethyl)-3-(4-(3-(piperidin-1-yl)cyclobutoxy)phenyl)urea;
BIOS-B-14: (R)-1-(2-(2-methylpyrrolidin-1-yl)ethyl)-3-(4-(3-(piperidin-1-yl)cyclobutoxy)phenyl)ure a;
BIOS-B-15: 1-(4-(3-(piperidin-1-yl)cyclobutoxy)phenyl)-3-(2-(piperidin-1-yl)ethyl)urea;
BIOS-B-16: 1-(2-(piperazin-1-yl)ethyl)-3-(4-(3-(piperidin-1-yl)cyclobutoxy)phenyl)urea;
BIOS-B-17:1-(2-(4-methyl-1,4-diazepan-1-yl)ethyl)-3-(4-(3-(piperidin-1-yl)cyclobutoxy)phenyl)ur ea;
BIOS-B-18: 1-(2-(4,4-difluoropiperidin-1-yl)ethyl)-3-(4-(3-(piperidin-1-yl)cyclobutoxy)phenyl)urea;
BIOS-B-19: 1-(2-(4-hydroxypiperidin-1-yl)ethyl)-3-(4-(3-(piperidin-1-yl)cyclobutoxy)phenyl)urea;
BIOS-B-20: 1-(4-(3-(piperidin-1-yl)cyclobutoxy)phenyl)-3-(2-thiomorpholinoethyl)urea;
BIOS-B-21: 1-(2-(2-(2-hydroxyethyl)piperidin-1-yl)ethyl)-3-(4-(3-(piperidin-1-yl)cyclobutoxy)phen yl)urea;
BIOS-B-22: 1-(2-(4-methylpiperazin-1-yl)ethyl)-3-(4-(3-(piperidin-1-yl)cyclobutoxy)phenyl)urea;
BIOS-B-23: 1-(2-(3-hydroxypiperidin-1-yl)ethyl)-3-(4-(3-(piperidin-1-yl)cyclobutoxy)phenyl)urea;
BIOS-B-24: 2-morpholino-N-(4-(3-(piperidin-1-yl)cyclobutoxy)phenyl)ethanethioamide;
BIOS-B-25: 2-morpholino-N-(4-(3-(piperidin-1-yl)cyclobutoxy)phenyl)ethanethioamide;
BIOS-B-26: (R)-2-(2-methylpyrrolidin-1-yl)-N-(4-(3-(piperidin-1-yl)cyclobutoxy)phenyl)ethanethioamide;
BIOS-B-27: 2-(piperidin-1-yl)-N-(4-(3-(piperidin-1-yl)cyclobutoxy)phenyl)ethanethioamide;
BIOS-B-28: 2-(4-methyl-1,4-diazepan-1-yl)-N-(4-(3-(piperidin-1-yl)cyclobutoxy)phenyl)ethanethioamide;
BIOS-B-29: 2-(diethylamino)-N-(4-(3-(piperidin-1-yl)cyclobutoxy)phenyl)acetamide;
BIOS-B-30: 2-(dimethylamino)-N-(4-(3-(piperidin-1-yl)cyclobutoxy)phenyl)acetamide;
BIOS-B-31: 2-(2-(dimethylamino)ethoxy)-N-(4-(3-(piperidin-1-yl)cyclobutoxy)phenyl)acetamide.

The compound as numbered in BIOS-B-1 to BIOS-B-31, or pharmaceutically acceptable salts thereof, may have the following structural formula:

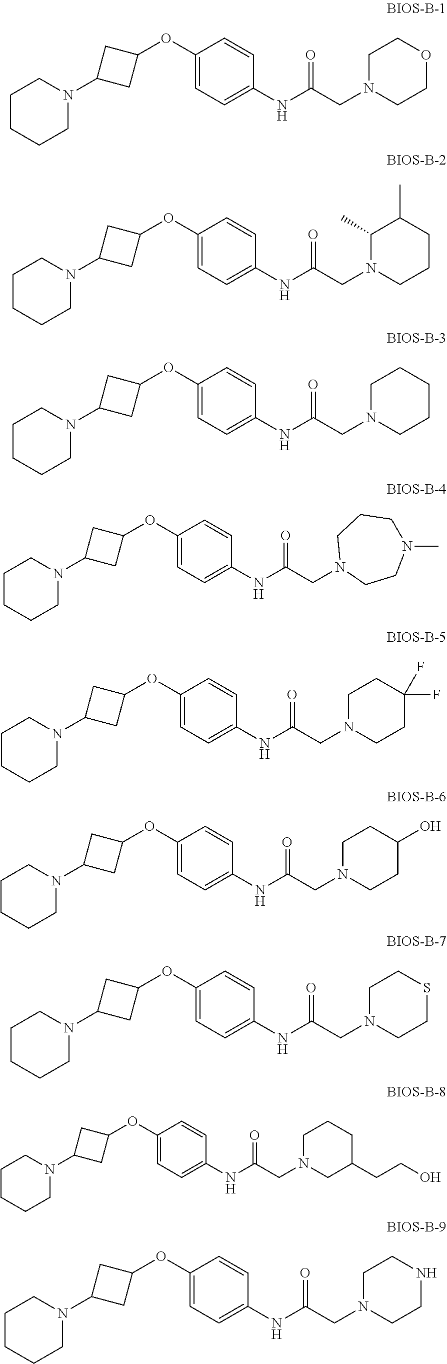

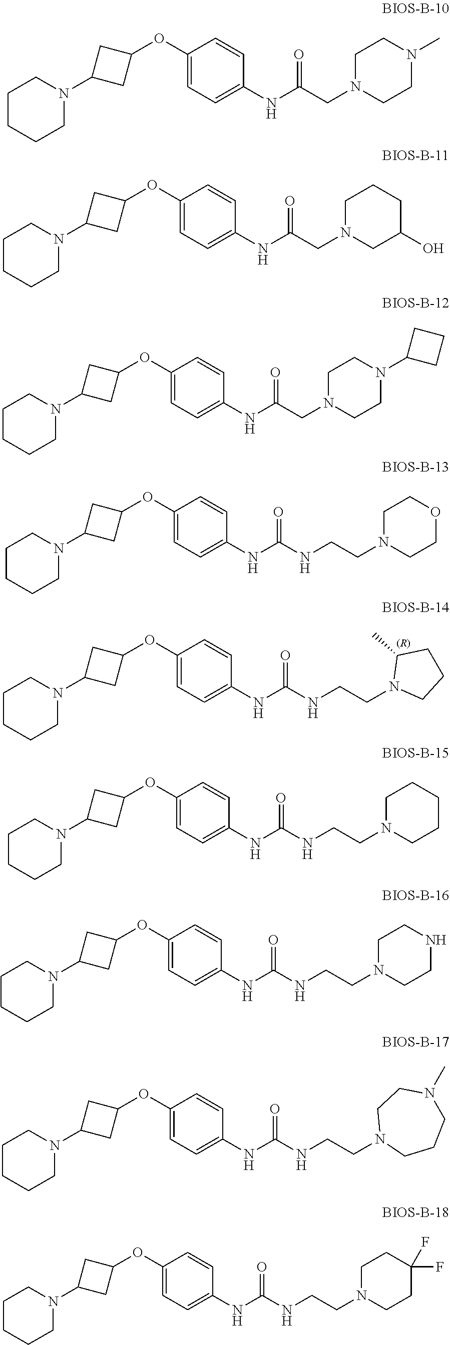

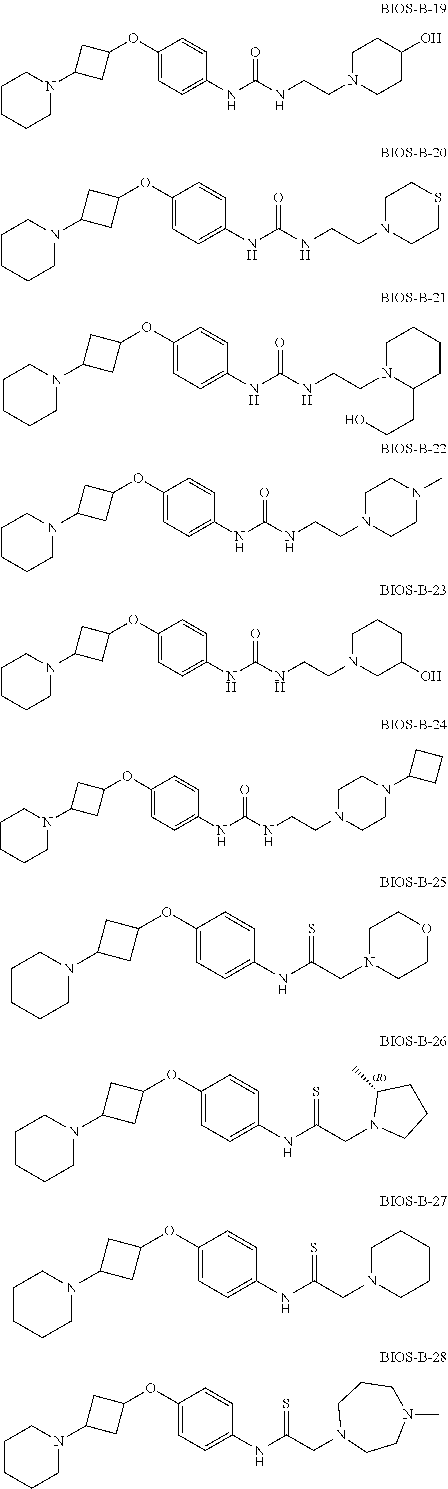

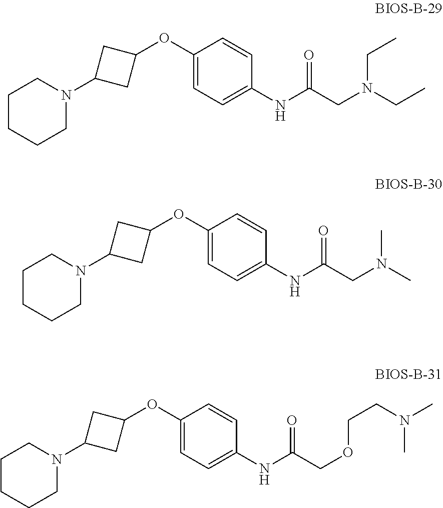 [SMILES: CCN(CC)CC(=O)Nc1ccc(OC2CC(N3CCCCC3)C2)cc1 ; CN(C)CC(=O)Nc1ccc(OC2CC(N3CCCCC3)C2)cc1 ; CN(C)CCOCC(=O)Nc1ccc(OC2CC(N3CCCCC3)C2)cc1]

Furthermore, the present invention also provides a histamine H3 receptor antagonist, which is a chiral compound as follows or a pharmaceutically acceptable salt thereof:
(R)-2-(2-methylpyrrolidin-1-yl)-N-(4-(3-(piperidin-1-yl)cyclobutoxy)phenyl)acetamide;
(R)-1-(2-(2-methylpyrrolidin-1-yl)ethyl)-3-(4-(3-(piperidin-1-yl)cyclobutoxy)phenyl)urea;
(R)-2-(2-methylpyrrolidin-1-yl)-N-(4-(3-(piperidin-1-yl)cyclobutoxy)phenyl)ethanethioamide.

The “compounds” described in the present invention include but are not limited to the following forms of compounds: free base, stereoisomers, geometric isomers, tautomers, isotopes, pharmaceutically acceptable salts, solvates, hydrates, prodrugs (esters or phosphates), etc.

The “compounds” described in the present invention can be asymmetric, for example, having one or more stereoisomers. Unless otherwise indicated, all stereoisomers are included, such as enantiomers and diastereomers. The compounds of the present invention containing asymmetric carbon atoms can be isolated in optically active pure form or in racemic form. Optically active pure forms can be obtained by splitting the racemic mixture, synthesizing it using chiral raw materials or chiral reagents.

The term “pharmaceutically acceptable salts” described in the present invention refers to salt of compounds in the present invention, prepared from compound has specific substituents with relatively non-toxic bases. When compounds contain relatively acidic functional groups, the bases addition salt of these compounds can be obtained by contacting the compound with a sufficient amount of base in pure solution or suitable inert solvents. Pharmaceutically acceptable bases addition salts, including, but not limited to, sodium, potassium, calcium, magnesium salts, ammonium or organic ammonia, such as alkali metal salts, alkaline earth metal salts, other metal salts, inorganic alkali salts, organic alkali salts, inorganic acid salts, lower alkane sulfonates, aryl sulfonates, organic acid salts, and amino acid salts.

In addition to the salt forms, the compounds provided herein exist in prodrug forms. The prodrugs of the compounds described herein are readily converted to the compounds of the present invention by chemical changes under physiological conditions. In addition, precursor drugs can be chemically or biochemically converted to the compounds of the present invention in vivo environment.

The compounds containing the above general structures, the terms used in this article have the following meanings:

The term “halogen” refers to fluorine, chlorine, bromine or iodine, preferably fluorine, chlorine or bromine.

The term “alkyl” refers to straight-chain or branched saturated hydrocarbon groups consisting of carbon atoms and hydrogen atoms, such as C₁₋₆ alkyl, including but not limited to methyl, ethyl, propyl (including n-propyl and isopropyl), butyl (including n-butyl, isobutyl, sec-butyl or tert-butyl), pentyl (including n-pentyl, isopentyl, neopentyl), n-hexyl, 2-methylhexyl.

The term “cycloalkyl” refers to monocyclic or bicyclic alkyl groups composed of carbon atoms and hydrogen atoms, such as a C₃₋₈ cycloalkyl group, including but not limited to cyclopropyl, cyclobutyl, cyclopentyl, cyclohexyl, cycloheptyl and cyclooctyl.

The term “alkoxy” refers to straight or branched alkyl groups connected by oxygen atoms, such as C₁₋₆ alkoxy, including but not limited to methoxy, ethoxy, n-propoxy (including n-propoxy and isopropoxy), butoxy (including n-butoxy, isobutoxy, sec-butoxy or tert-butoxy), pentoxy (including n-pentoxy, isopentoxy, neopentoxy), n-hexyloxy, 2-methylhexyloxy, etc.

The term “alkylamino” refers to open-chain alkyl groups containing nitrogen atoms, such as C₁₋₆ alkylamino, including but not limited to methylamino, ethylamino, isopropylamino, dimethylamino, methylethylamino, diethylamino, etc.

The term “aryl” refers to all-carbon monocyclic or condensed polycyclic groups of 5-12 (integer) carbon atoms, having a completely conjugated n-electron system, including but not limited to benzene rings, naphthalene rings, and anthracene rings.

The term “heterocyclic group” or “heteroaryl” refers to monocyclic or condensed rings having 3-12 (integer) ring atoms, wherein 1, 2, 3 or more ring atoms are one or more selected from N, O, and S, and the remaining ring atoms are C, and have a completely conjugated or unconjugated n-electron system. The heterocyclic group can be saturated or unsaturated groups. Examples of heterocyclic groups include, but are not limited to, pyrrolyl, indolyl, pyrrolidinyl, imidazolyl, pyrazolyl, tetrazolyl, pyridinyl, quinolyl, isoquinolyl, piperidinyl, pyrimidinyl, pyrazinyl, piperazinyl, furanyl, pyranyl, and morpholinyl.

The invention provides a method of preparing the above compounds by the following steps, but not limited to the following methods:

### General Synthesis Method 1: Refer to General Formula II, where X═O, R₁═H, R₀═NR′R″

The reaction route is as follows:

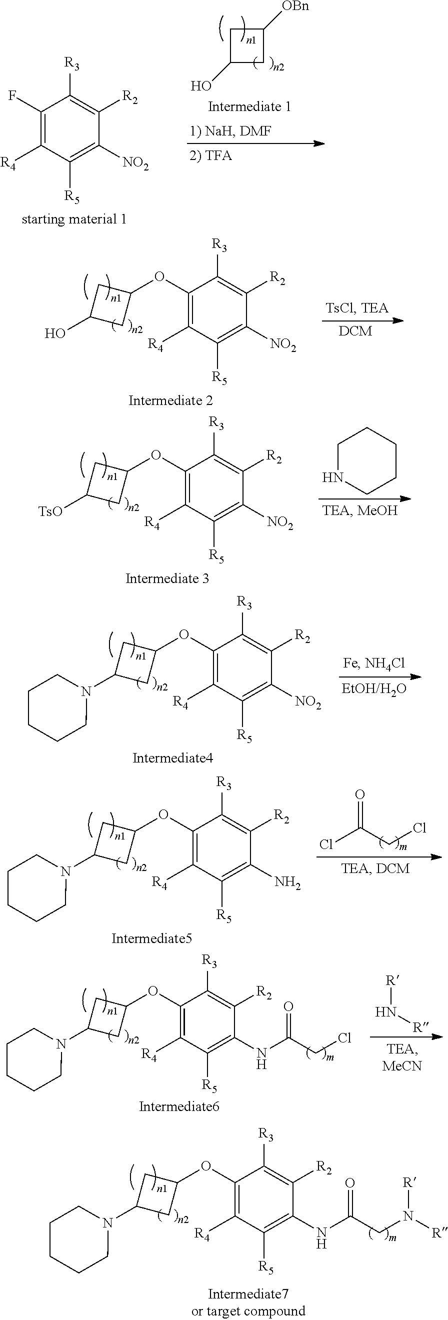

The corresponding reaction procedure is shown as follows:

The starting material 1 (p-fluoronitrobenzene analogs) reacts with Intermediate 1 and sodium hydride dissolved in N,N-dimethylformamide, and then reactes in trifluoroacetic acid to obtain intermediate 2. Intermediate 2 then reactes with p-toluenesulfonyl chloride to obtain intermediate 3. Intermediate 3 reats with piperidine via SN₂ to obtain intermediate 4. Intermediate 4 is reduced to intermediate 5 using ammonium chloride as hydrogen source under Fe catalysis. Intermediate 5 is acylated by halogenated alkyl chloride to intermediate 6, which undergoes reductive amination to obtain intermediate 7 or the target compounds.

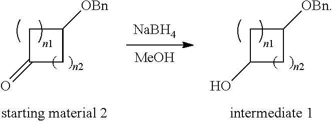

The corresponding preparation route of Intermediate 1 is shown as follows:

### General Synthesis Method 2: Refer to General Formula III, where X═O, R₁═H, R₆═H, R₀═NR′R″

The corresponding preparation route is shown as follows:

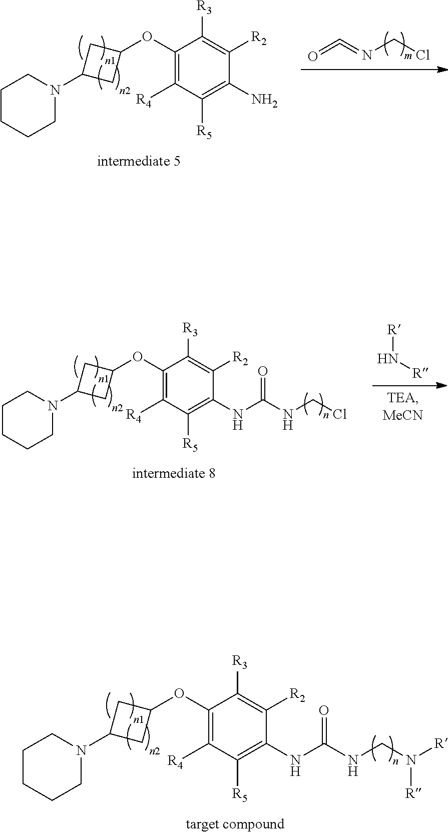

The corresponding preparation process is shown as follows:

Intermediate 5 reacts with chloroalkyl isocyanate to form intermediate 8, which is substituted with amine to afford the target compounds.

### General Synthesis Method 3: Refer to General Formula II, where X═S, R₁═H, R₀═NR′R″

The corresponding preparation route is shown as follows:

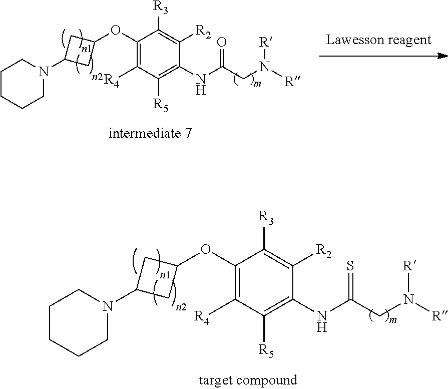

The reaction process is as follows: intermediate 7 is reacted with Lawesson's reagent to obtain the target compounds.

The substituents R′, R″, R₁, R₂, R₃, R₄, and R₅ involved in the aforementioned synthesis method 1, synthesis method 2, and synthesis method 3 are defined as follows: R′ and R″ are each independently selected from hydrogen, C₁₋₆ alkyl, C₁₋₆ alkoxy, halogen, amino, hydroxy, carboxyl, carbonyl, amide, cyano, C₁₋₆ haloalkyl, C₁₋₆ alkylhydroxy, C₁₋₆ alkylamino, C₁₋₆ alkylamide, C₁₋₆ haloalkoxy, C₁₋₆ alkoxyhydroxy, C₁₋₆ alkoxyamino, and C₁₋₆ alkoxyamide, and R′ and R″ are not hydrogen at the same time; R₂, R₃, R₄, and R₅ are each independently one or more selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆alkylamino, C₁₋₆ alkylamide, C₁₋₆ alkylsulfone, and C₁₋₆ alkylsulfoxide; Or, R₂, R₃ and two carbon atoms on the benzene ring, to which R₂, R₃ are connected respectively, form a substituted or unsubstituted benzobicyclic structure, wherein the benzobicyclic structure may be a benzoheterocyclic ring, but is not limited thereto; Or, R₄, R₅ and the two carbon atoms on the benzene ring to which R₄, R₅ are connected respectively, form a substituted or unsubstituted benzobicyclic structure; wherein the benzobicyclic structure may be a benzoheterocyclic ring, but is not limited thereto.

The present invention also provides a pharmaceutical composition comprising at least one compound as hereinbefore described or a pharmaceutically acceptable salt thereof as an active ingredient, and at least one or more pharmaceutically acceptable carriers.

The term “pharmaceutical composition” as used in the invention refers to a formulation of one or more compounds of the present invention or salts thereof with a carrier commonly accepted in the art for the delivery of biologically active compounds to an organism (e.g., human beings). The pharmaceutical compositions are intended to facilitate drug delivery to the organism.

The routes of administration of the compounds or pharmaceutically acceptable salts thereof, or pharmaceutical compositions thereof described herein, include, but are not limited to, oral, rectal, transmucosal, trans-intestinal, topical, transdermal, inhalation, parenteral, sublingual, intravaginal, intranasal, intraocular, intraperitoneal, intramuscular, subcutaneous, and intraventricular administration. The preferred route of administration is oral administration.

The present invention also provides uses of preparing compounds or pharmaceutical compositions as described hereinbefore in the prevention or treatment of diseases associated with histamine H3 receptors.

Preferably, the foregoing medications are used for the prevention or treatment of cognitive disorders, dementia, attention deficit hyperactivity disorder, schizophrenia, epilepsy, sleep disorders, sleep apnea, obesity, eating disorders, pain and pruritus.

Preferably, the foregoing medications are used for the prevention or treatment of neuropathic pain, including but not limited to peripheral neuropathic pain or central neuropathic pain.

Preferably, the peripheral neuropathic pain is trigeminal neuralgia, glossopharyngeal neuralgia, acute or chronic inflammatory demyelinating polyneuropathic neuralgia, alcoholic polyneuralgia, chemotherapy-induced polyneuropathic neuralgia, complex regional pain syndrome, embedded compression neuralgia (e.g., carpal tunnel syndrome), HIV sensory neuralgia, medically induced neuralgia (e.g., post-mastectomy pain), tumor compression or infiltration neuralgia, nutritional deficiency-associated neuralgia, diabetic neuralgia, phantom limb pain, postherpetic neuralgia, post-radiation plexopathy, radiculopathy (cervical, thoracic, or lumbosacral), toxic exposure-associated neuralgia, or post-traumatic neuralgia.

Preferably, the central neuropathic pain is post-stroke pain, multiple sclerosis-associated pain, Parkinson's disease-associated pain, post-traumatic spinal cord injurious pain, spinal cord cavernous disease, post ischemic myelopathy, compressive myelopathy, HIV myelopathy, or post-radiation myelopathy.

Compared with the prior arts, the present invention has the following beneficial effects:

The present invention has developed a series of structurally novel histamine H3 receptor antagonists based on histamine H3 receptor ligands. The series of compounds are characterized by simple preparation routes and easy availability of raw materials. The results of the series of related biological tests all indicate that the compounds have significant H3 receptor antagonistic activity and can be used as lead compounds to prevent or treat a disease associated with histamine H3 receptor.

### DESCRIPTION OF THE DRAWINGS

FIG. 1 shows the graphs of the changes in mechanical tingling thresholds of each experimental group at different times and different doses. In the graph, compared with the MODEL group, *, P<0.05; **, P<0.01; compared with the SHAM group, #, P<0.05; ##, P<0.01.

FIG. 2 shows the change of mechanical sting thresholds of each experimental group at different times. In the graph, compared with the MODEL group, *, P<0.05; **, P<0.01; compared with the SHAM group, #, P<0.05; ##, P<0.01.

### EMBODIMENTS OF THE INVENTION

The following are specific embodiments of the present invention to further describe the technical solution, but the protection scope of the present invention is not limited to these embodiments. Any changes or equivalent substitutions that do not deviate from the concept of the present invention are included in the protection scope of the present invention.

The solvents, equipment, codes and full names involved in the embodiments of the present invention are as follows:
Code Bn: refers to benzyl;
Code DMF: refers to N,N-dimethylformamide;
Code TFA: refers to trifluoroacetic acid;
Code TsCI: refers to p-Toluenesulfonyl chloride;
Code TEA: refers to triethylamine;
Code DCM: refers to dichloromethane;
Code Boc: refers to tert-Butyloxycarbonyl;
Code Lawesson reagent: 2,4-Bis(p-methoxyphenyl)-1,3-dithiodiphosphatane-2,4-sulfide.

The preparation methods of the target compounds in the present invention: the liquid chromatography uses Waters Symmetry C18 chromatographic columns; TLC is performed using GF254 (0.25 mm); Nuclear magnetic resonance spectroscopy (NMR) is measured by Bruker-400 NMR spectrometers; Liquid chromatography-mass spectrometry (LC/MS) is performed by Waters ZQ mass spectrometer detector (column: Waters Symmetry C18, mm, 5 μm, 35° C.) in ESI (+) ion mode.

In addition, all operations involving easily oxidized or hydrolyzed starting materials are carried out under nitrogen protection. Unless otherwise specified, the raw materials used in the present invention are commercially available and can be used directly without further purification.

The starting materials, common intermediates, etc. involved in the embodiments of the present invention can be purchased commercially or obtained by self-production. Among them, the starting materials and common intermediates that need to be obtained by self-production are prepared in detail as follows.

### I. Preparation of Starting Material 2:

The starting material 2 involved in the preparation process of the intermediate material B1 is synthesized by the applicant, and the synthesis route is as follows:

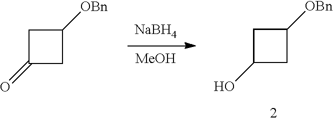 [SMILES: "MeOH" ; O=C1CC(OCc2ccccc2)C1 ; "NaBH4" ; OC1CC(OCc2ccccc2)C1]

To a 250 mL bottle added 3-(benzyloxy)cyclobutanone (5.0 g, 28.37 mmol) and anhydrous methanol (34 mL) under Nitrgen atmosphere. The reaction was cooled by ice for 15 minutes before sodium borohydride added in batches (717 mg/batch, 3 batches, 56.75 mmol) during 0.5 hours, and finally the reaction was transferred to room temperature for 4-5 hours. When the reaction was completed, the solution was quenched with 1.0 mL of saline, and separated with ethyl acetate twice. The organic phase was combined and washed by saline, dried over by anhydrous sodium sulfate, and finally concentrated to dryness obtaining 5.31 g of colorless oil (compound 2) yielding 99%.

MS: 179.1[M+H]+.

¹H NMR (400 MHz, DMSO-d₆) δ 7.38˜7.26 (m, 5H), 4.48 (s, 2H), 4.04 (t, J=5.6 Hz, 1H), 3.92˜3.79 (m, 1H), 3.80 (d, J=4.4 Hz, 1H), 2.35˜2.30 (m 2H), 2.03˜1.99 (m, 2H).

### II. Preparation of Intermediate Material B1:

The synthesis route of intermediate material B1 is as follows:

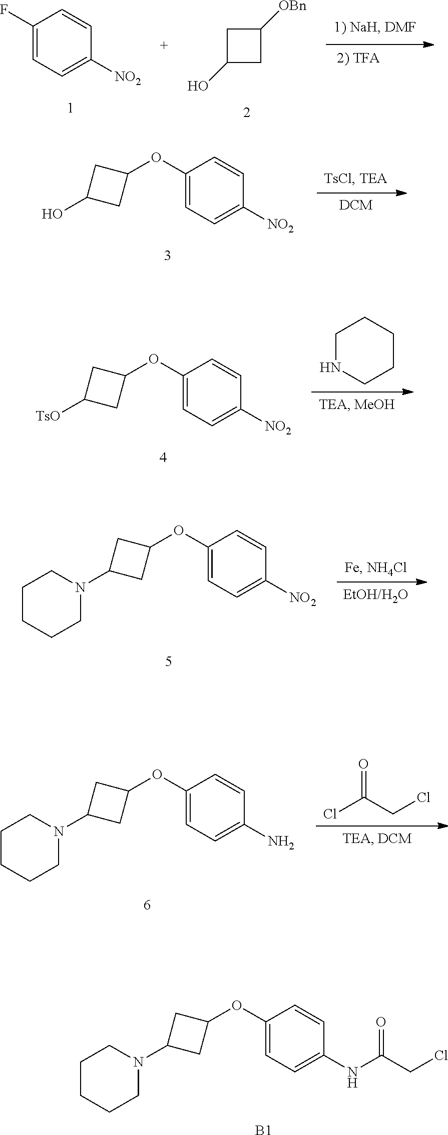

### Step 1: Synthesis of 3-(4-nitrophenoxy)cyclobutanol (3)

Compound 2 (2.155 g, 12.09 mmol) was dissolved in 15 mL anhydrous N,N-dimethylformamide, and cooled by ice for 5 minutes. Sodium hydride (580 mg, 14.51 mmol) was added under nitrogen atmosphere and reacted in ice bath for 0.5-1 hour. A solution of 4-fluoronitrobenzene (compound 1, 1.54 g) in 5 mL anhydrous N,N-dimethylformamide was added slowly dropwise within 15 minutes. The reaction was carried out at room temperature for 5-6 hours. After the reaction completed, the reaction was quenched by 200 mL ice water, extracted by ethyl acetate (50 mL×3), the organic phase was combined, washed by water, saline, and dried with anhydrous sodium sulfate, concentrated to obtain 3.89 g yellow crude product yielding 99%.

The yellow crude (3.89 g) was dissolved in 40 mL trifluoroacetic acid and reluxed for 3-4 hours. When the reaction was completed, the reaction was cooled to 0° C. by ice water bath. Sodium hydroxide solution was added dropwise to adjust the pH to neutral, extracted by ethyl acetate (70 mL×3), and the organic phases were combined. The organic phase was washed by saline, dried over anhydrous sodium sulfate, and concentrated to obtain 4.4 g crude yellow produce. Rapid column chromatography purification (PE:EA=2:1) yielded 1.358 g light yellow powder (compound 3) with a yield of 53.7%.

MS: 210.1[M+H]⁺.

¹H NMR (400 MHz, DMSO-d₆) δ 8.19 (d, J=9.2 Hz, 2H), 7.05 (d, J=9.2 Hz, 2H), 5.26 (d, J=6.8 Hz, 1H), 4.43 (m, 1H), 3.87 (m, 1H), 2.91˜2.83 (m, 2H), 2.01˜1.87 (m, 2H).

### Step 2: Synthesis of 3-(4-nitrophenoxy)cyclobutyl 4-methylbenzenesulfonate (4)

Compound 3 (4.64 g, 22.22 mmol), 9.2 mL triethylamine and p-toluenesulfonic chloride (1.86 g, 33.33 mmol) were dissolved in 40 mL anhydrous dichloromethane under nitrogen atmosphere. The mixture was transferred to oil bath and reacted overnight under 30° C. When the reaction completed, the reaction was quenched with 20 mL water, the aqueous phase was extracted with dichloromethane (50 mL×3). The organic phase was combined, washed with saline, dried with anhydrous sodium sulfate, and concentrated to obtain the crude product. Finally, the crude was purified and crystallized by methyl tert butyl ether obtaining 6.73 g white powder (compound 4) with a yield of 83.38%.

MS: 364.1[M+H]⁺.

¹H NMR (400 MHz, DMSO-d₆) δ 8.17 (s, 2H), 7.82 (d, J=9.0 Hz, 2H), 7.50 (d, J=9.0 Hz, 2H), 7.00 (d, J=7.8, 2H), 4.62 (t, J=7.0 Hz, 1H), 4.50 (t, J=7.0 Hz, 1H), 2.94˜2.87 (m, 2H), 2.43 (s, 3H), 2.22˜2.15 (m, 2H).

### Step 3: Synthesis of 1-(3-(4-nitrophenoxy)cyclobutyl)piperidine (5)

Compound 4 (800 mg, 2.20 mmol) was mixed with 10 mL piperidine, reached at 100° C. overnight. When the reaction completed, the reaction was cooled to room temperature, quenched with 20 mL water, and then extracted with ethyl acetate (50 mL×3). The organic phase was combined and washed with saline, dried over with anhydrous sodium sulfate, and concentrated to obtain 736 mg crude (compound 5), yielding 99%.

MS: 277.1[M+H]⁺.

¹H NMR (400 MHz, DMSO-d₆) δ 8.28˜8.14 (d, 2H), 7.11˜6.98 (d, 2H), 4.90˜4.85 (m, 1H), 2.91˜2.86 (m, 1H), 2.91˜2.86 (m, 3H), 2.28˜2.14 (m, 5H), 1.55˜1.30 (m, 10H).

### Step 4: Synthesis of 4-(3-(piperidin-1-yl)cyclobutoxy)aniline (6)

Compound 5 (736 mg), reduced iron powder (862 mg, 7.0 equiv.) and ammonium chloride (214 mg, 3.96 mmol) were dissolved in 6 mL ethanol and 2 mL water. The reaction was carried out at 80° C. for 1-2 hours. When the reaction was completed, the reaction was cooled to room temperature, filtered. The filtrate was concentrated to dryness, and 10 mL sodium bicarbonate solution was added, and then extracted with ethyl acetate (20 mL×3). The organic phase was combined, washed with saline, dried over with anhydrous sodium sulfate, and concentrated to obtain 583 mg brown oil (compound 6), yielding 99%.

MS: 347.2[M+H]⁺.

### Step 5: Synthesis of 2-chloro-N-(4-(3-(piperidin-1-yl)cyclobutoxy)phenyl)acetamide (B1)

Compound 6 (852 mg, 3.46 mmol) and triethylamine (961 μL, 6.92 mmol) were dissolved in 12 mL anhydrous dichloromethane. 358 μL chloroacetyl chloride was added and allowed to react overnight at room temperature. When the reaction was completed, the reaction was quenched with 15 mL sodium bicarbonate solution, extracted with dichloromethane (20 mL×3). The organic phase was combined and washed with saline, dried with anhydrous sodium sulfate, and concentrated to obtain 1.42 g crude. Rapid column chromatography purification (PE:EA:TEA=2:1:0.2) yielded 720 mg light yellow powder (compound B1), yielding 64.5%.

MS: 352.3[M+H]⁺.

¹H NMR (400 MHz, DMSO-d₆) δ 10.14 (s, 1H), 7.47 (d, 2H), 6.78 (d, 2H), 4.70˜4.65 (m, 1H), 4.21 (s, 2H), 2.91˜2.79 (m, 1H), 2.37˜2.05 (m, 8H), 1.55˜1.32 (m, 6H).

### II. Preparation of Intermediate Material B2:

The synthesis route of intermediate material B2 is as follows:

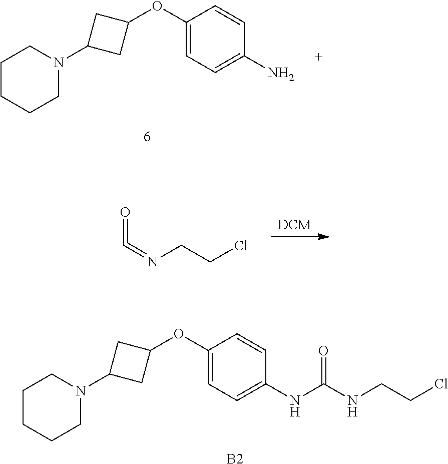

Compound 6 (1.896 g, 7.70 mmol) was dissolved in 30 mL Anhydrous dichloromethane. 788 μL chloroehyl isocyanate was added at −5° C., and the reaction was finally transferred to room temperature for 5-6 hours (with large amount of white precipitate appeared). When the reaction completed, the reaction solution was filtered, the filter was dried to afford 1.894 g white powder (compound B2), yielding 73.3%.

MS: 352.3[M+H]⁺.

¹H NMR (400 MHz, DMSO-d₆) δ 8.42 (s, 1H), 7.26 (d, J=9.0 Hz, 2H), 6.69 (d, J=9.0 Hz, 2H), 6.28 (t, J=5.8 Hz, 1H), 4.66˜4.61 (m, 1H), 3.64 (t, J=6.2 Hz, 2H), 3.40 (q, J=6.2 Hz, 2H), 2.85 (s, 1H), 2.40˜1.98 (m, 8H), 1.60˜1.25 (m, 6H).

The following Example 1 to 31 are the preparation examples of the free base and/or hydrochloride structure of the BIOS-S-series compounds described in the present invention. It should be noted that in addition to the hydrochloride from mentioned in the Examples, BIOS-B series compounds can also be prepared in other pharmaceutically acceptable salt forms, including but not limited to sulfates, nitrates, nitrites, maleic acid salts, fumarates, formatted, diformes, acetates, etc. The preparation of these compounds can be inspired by the corresponding technical insights provided in Examples 1 to 31 of the present invention.

### Example 1: Synthesis of 2-morpholino-N-(4-(3-(piperidin-1-yl)cyclobutoxy)phenyl)acetamide (BIOS-B-1)

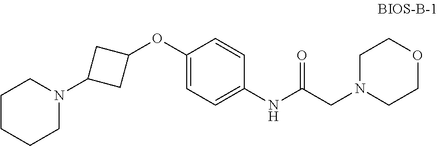 [SMILES: O=C(CN1CCOCC1)Nc1ccc(OC2CC(N3CCCCC3)C2)cc1]

The synthesis route is as follows:

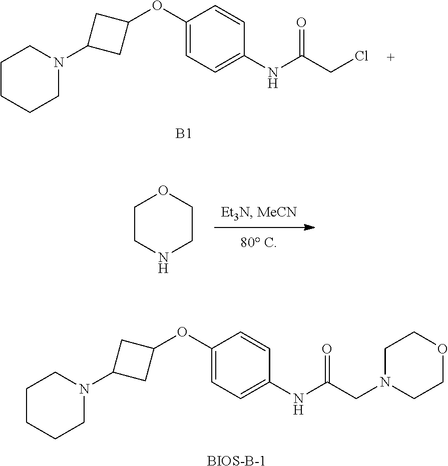 [SMILES: O=C(CCl)Nc1ccc(OC2CC(N3CCCCC3)C2)cc1 ; "B1" ; C1COCCN1 ; O=C(CN1CCOCC1)Nc1ccc(OC2CC(N3CCCCC3)C2)cc1 ; "BIOS-B-1"]

Intermediate B1 (100 mg, 0.310 mmol), morpholine (81 μL, 0.929 mmol), and triethylamine 323 μL were dissolved in 4 mL acetonitrile and transferred to react overnight at 80° C. When the reaction completed, Concentrate the reaction solution, and purified by Thin layer chromatography (PE:EA:TEA=5:4:1), affording 81 mg light brown oil (compound BIOS-B-1), yielding 69.96%.

Furthermore, the hydrochloride of compound BIOS-B-1 could be prepared by reacting with HCl/EA for 1-2 hours, which afford 86 mg white solid with a yield of 88.83%.

MS: 374.1[M+H]⁺.

¹H NMR (400 MHz, DMSO-d₆) δ 9.58 (s, 1H), 7.50 (d, J=9.0 Hz, 2H), 6.75 (d, J=9.0 Hz, 2H), 4.69˜4.64 (m, 1H), 3.70˜3.54 (m, 4H), 3.08 (s, 2H), 2.88˜2.81 (m, 1H), 2.50˜2.46 (m, 4H), 2.37˜2.00 (m, 8H), 1.60˜1.35 (m, 6H).

### Example 2: Synthesis of (R)-2-(2-methylpyrrolidin-1-yl)-N-(4-(3-(piperidin-1-yl)cyclobutoxy) phenyl)acetamide (BIOS-B-2)

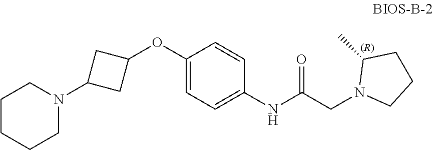

The synthesis route is as follows:

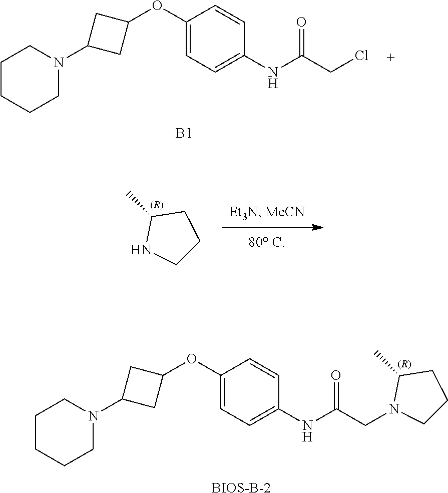

Referred to the synthesis route of compound BIOS-B-1 in Example 1, (R)-2-methylpyrrolidine from this example was used instead of morpholine from Example 1, and the remaining preparation steps were the same as in Example 1. 75 mg yellow brown oil (compound BIOS-B-2) was obtained with a yield of 65.12%.

Furthermore, the hydrochloride of compound BIOS-B-2 could be prepared by reacting with HCl/EA for 1-2 hours, which afford 71 mg pink solid with a yield of 79.13%.

MS: 372.1[M+H]⁺.

¹H NMR (400 MHz, DMSO-d₆) δ 9.47 (s, 1H), 7.50 (d, J=9.0 Hz, 2H), 6.75 (d, J=9.0 Hz, 2H), 4.69˜4.64 (m, 1H), 3.38 (d, J=15.5 Hz, 1H), 3.11˜3.06 (m, 1H), 2.95 (d, J=15.5 Hz, 1H), 2.88˜2.81 (m, 1H), 2.49˜2.03 (m, 10H), 1.97˜1.87 (m, 1H), 1.81˜1.60 (m, 2H), 1.57˜1.29 (m, 7H), 1.06 (d, J=6.1 Hz, 3H).

### Example 3: Synthesis of 2-(piperidin-1-yl)-N-(4-(3-(piperidin-1-yl)cyclobutoxy)phenyl)acetamide (BIOS-B-3)

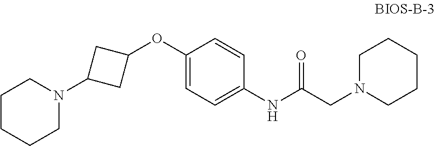 [SMILES: O=C(CN1CCCCC1)Nc1ccc(OC2CC(N3CCCCC3)C2)cc1]

The synthesis route is as follows:

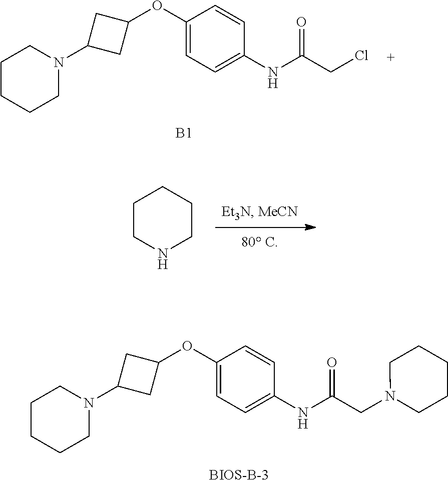 [SMILES: O=C(CCl)Nc1ccc(OC2CC(N3CCCCC3)C2)cc1 ; "B1" ; C1CCNCC1 ; O=C(CN1CCCCC1)Nc1ccc(OC2CC(N3CCCCC3)C2)cc1 ; "BIOS-B-3"]

Intermediate B1 (100 mg, 0.310 mmol), morpholine (81 μL, 0.929 mmol), and triethylamine 323 μL were dissolved in 4 mL acetonitrile and transferred to react overnight at 80° C. When the reaction completed, Concentrate the reaction solution, and purified by Thin layer chromatography (PE:EA:TEA=5:4:1), affording 100 mg light yellow solid (compound BIOS-B-3), yielding 86.82%.

MS: 372.1[M+H]⁺.

¹H NMR (400 MHz, DMSO-d₆) δ 9.50 (s, 1H), 7.50 (d, J=9.0 Hz, 2H), 6.75 (d, J=9.0 Hz, 2H), 4.70˜4.65 (m, 1H), 3.02 (s, 2H), 2.95˜2.81 (m, 1H), 2.44 (t, J=5.3 Hz, 4H), 2.38˜2.03 (m, 8H), 1.66˜1.31 (m, 12H).

### Example 4: Synthesis of 2-(4-methyl-1,4-diazepan-1-yl)-N-(4-(3-(piperidin-1-yl)cyclobutoxy) phenyl)acetamide (BIOS-B-4)

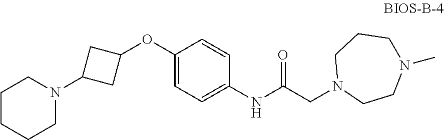 [SMILES: CN1CCCN(CC(=O)Nc2ccc(OC3CC(N4CCCCC4)C3)cc2)CC1]

The synthesis route is as follows:

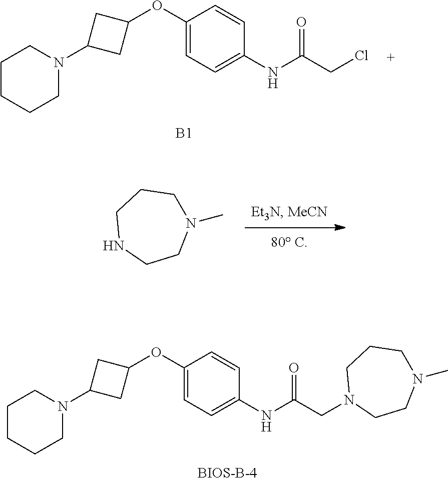

Referred to the synthesis route of compound BIOS-B-1 in Example 1, 1-methyl-1,4-diazepane from this example was used instead of morpholine from Example 1, and the remaining preparation steps were the same as in Example 1. 92 mg yellow solid (compound BIOS-B-4) was obtained with a yield of 74.09%.

Furthermore, the hydrochloride of compound BIOS-B-4 could be prepared by reacting with HCl/EA for 1-2 hours, which afford 76 mg light yellow solid with a yield of 64.89%.

MS: 401.1[M+H]⁺.

¹H NMR (400 MHz, DMSO-d₆) δ 9.53 (s, 1H), 7.50 (d, J=9.0 Hz, 2H), 6.75 (d, J=9.0 Hz, 2H), 4.70˜4.64 (m, 1H), 3.23 (s, 2H), 2.87˜2.83 (m, 1H), 2.80˜2.71 (m, 4H), 2.64˜2.56 (m, 4H), 2.38˜2.14 (m, 9H), 2.12˜2.06 (m, 2H), 1.83˜1.71 (m, 2H), 1.56˜1.32 (m, 6H).

### Example 5: Synthesis of 2-(4,4-difluoropiperidin-1-yl)-N-(4-(3-(piperidin-1-yl)cyclobutoxy)phenyl)acetamide (BIOS-B-5)

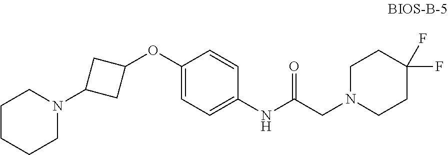 [SMILES: O=C(CN1CCC(F)(F)CC1)Nc1ccc(OC2CC(N3CCCCC3)C2)cc1]

The synthesis route is as follows:

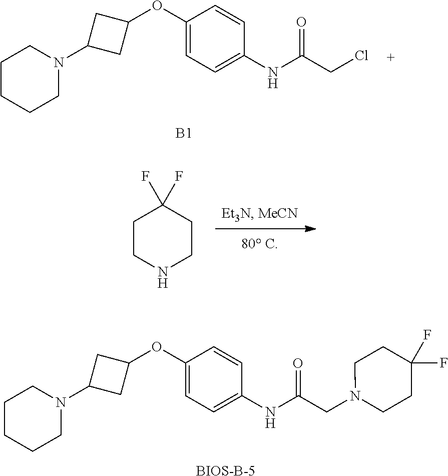

Referred to the synthesis route of compound BIOS-B-3 in Example 3, 4,4-difluoropiperidine from this example was used instead of piperidine from Example 3, and the remaining preparation steps were the same as in Example 3. 61 mg light yellow solid (compound BIOS-B-5) was obtained with a yield of 69.66%.

MS: 408.1[M+H]⁺.

¹H NMR (400 MHz, DMSO-d₆) δ 9.59 (s, 1H), 7.51 (d, J=9.0 Hz, 1H), 6.75 (d, J=9.0 Hz, 2H), 4.70˜4.65 (m, 1H), 3.17 (s, 2H), 2.87 (s, 1H), 2.64 (t, J=5.6 Hz, 4H), 2.40˜1.95 (m, 12H), 1.57˜1.31 (m, 6H).

### Example 6: Synthesis of 2-(4-hydroxypiperidin-1-yl)-N-(4-(3-(piperidin-1-yl)cyclobutoxy)phenyl)acetamide (BIOS-B-6)

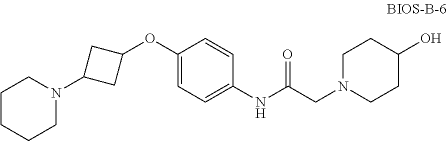 [SMILES: O=C(CN1CCC(O)CC1)Nc1ccc(OC2CC(N3CCCCC3)C2)cc1]

The synthesis route is as follows:

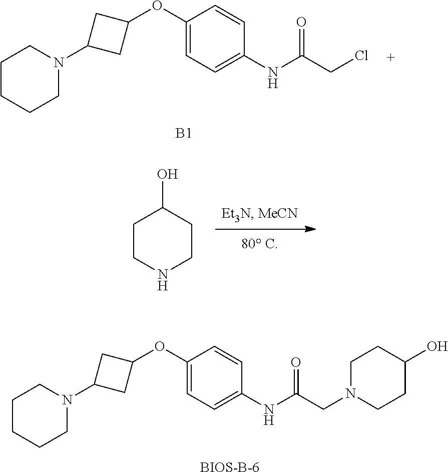 [SMILES: O=C(CCl)Nc1ccc(OC2CC(N3CCCCC3)C2)cc1 ; "B1" ; OC1CCNCC1 ; O=C(CN1CCC(O)CC1)Nc1ccc(OC2CC(N3CCCCC3)C2)cc1 ; "BIOS-B-6"]

Referred to the synthesis route of compound BIOS-B-1 in Example 1, 4-hydroxypyridine from this example was used instead of morpholine from Example 1, and the remaining preparation steps were the same as in Example 1. 94 mg yellow solid (compound BIOS-B-6) was obtained with a yield of 78.25%.

Furthermore, the hydrochloride of compound BIOS-B-6 could be prepared by reacting with HCl/EA for 1-2 hours, which afford 99 mg white solid with a yield of 88.64%.

MS: 388.1[M+H]⁺.

¹H NMR (400 MHz, DMSO-d₆) δ 9.51 (s, 1H), 7.50 (d, J=9.0 Hz, 2H), 6.75 (d, J=9.0 Hz, 2H), 4.70˜4.65 (m, 1H), 4.58 (d, J=4.0 Hz, 1H), 3.50˜3.45 (m, 1H), 3.04 (s, 2H), 2.91 (s, 1H), 2.76˜2.71 (m, 2H), 2.40˜2.03 (m, 10H), 1.80˜1.68 (m, 2H), 1.60˜1.32 (m, 8H).

### Example 7: Synthesis of N-(4-(3-(piperidin-1-yl)cyclobutoxy)phenyl)-2-thiomorpholinoacetamide (BIOS-B-7)

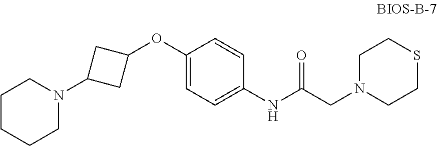 [SMILES: O=C(CN1CCSCC1)Nc1ccc(OC2CC(N3CCCCC3)C2)cc1]

The synthesis route is as follows:

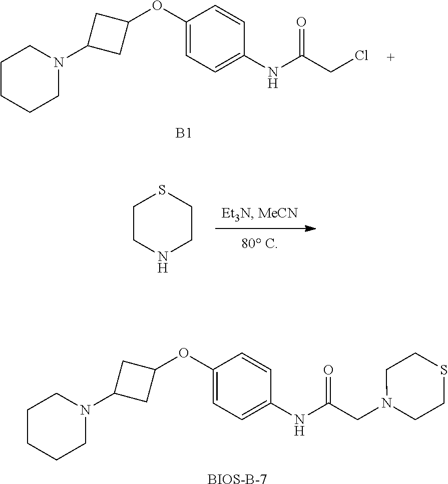 [SMILES: O=C(CCl)Nc1ccc(OC2CC(N3CCCCC3)C2)cc1 ; "B1" ; C1CSCCN1 ; O=C(CN1CCSCC1)Nc1ccc(OC2CC(N3CCCCC3)C2)cc1 ; "BIOS-B-7"]

Referred to the synthesis route of compound BIOS-B-3 in Example 3, Thiomorpholine from this example was used instead of piperidine from Example 3, and the remaining preparation steps were the same as in Example 3. 86 mg light yellow solid (compound BIOS-B-7) was obtained with a yield of 71.21%.

MS: 390.0[M+H]⁺.

¹H NMR (400 MHz, DMSO-d₆) δ 9.51 (s, 1H), 7.51 (d, J=9.0 Hz, 2H), 6.75 (d, J=9.0 Hz, 2H), 4.70˜4.65 (m, 1H), 3.10 (s, 2H), 2.87 (s, 1H), 2.77˜2.74 (m, 4H), 2.73˜2.63 (m, 4H), 2.40˜2.04 (m, 8H), 1.61˜1.32 (m, 6H).

### Example 8: Synthesis of 2-(3-(2-hydroxyethyl)piperidin-1-yl)-N-(4-(3-(piperidin-1-yl)cyclobutoxy)phenyl)acetamide (BIOS-B-8)

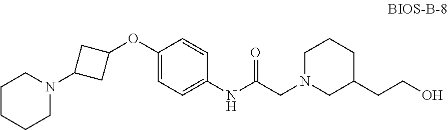 [SMILES: O=C(CN1CCCC(CCO)C1)Nc1ccc(OC2CC(N3CCCCC3)C2)cc1]

The synthesis route is as follows:

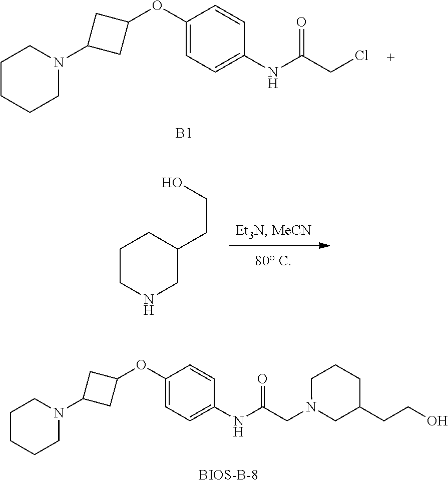 [SMILES: O=C(CCl)Nc1ccc(OC2CC(N3CCCCC3)C2)cc1 ; "B1" ; OCCC1CCCNC1 ; O=C(CN1CCCC(CCO)C1)Nc1ccc(OC2CC(N3CCCCC3)C2)cc1 ; "BIOS-B-8"]

Referred to the synthesis route of compound BIOS-B-1 in Example 1, 2-piperidine ethanol from this example was used instead of morpholine from Example 1, and the remaining preparation steps were the same as in Example 1. 105 mg colorless oil (compound BIOS-B-8) was obtained with a yield of 81.50%.

Furthermore, the hydrochloride of compound BIOS-B-8 could be prepared by reacting with HCl/EA for 1-2 hours, which afford 101 mg white solid with a yield of 81.83%.

MS: 416.1[M+H]⁺.

¹H NMR (400 MHz, DMSO-d₆) δ 9.53 (s, 1H), 7.50 (d, J=9.1 Hz, 1H), 6.76 (d, J=9.1 Hz, 2H), 4.70˜4.65 (m, 1H), 4.46 (s, 1H), 3.57˜3.38 (m, 2H), 3.26 (d, J=16.4 Hz, 1H), 3.05 (d, J=16.4 Hz, 1H), 2.93˜2.70 (m, 2H), 2.59˜2.55 (m, 1H), 2.46˜2.16 (m, 7H), 2.13˜2.08 (m, 2H), 1.77˜1.62 (m, 3H H), 1.54˜1.45 (m, 7H), 1.44˜1.26 (m, 4H).

### Example 9: Synthesis of 2-(piperazin-1-yl)-N-(4-(3-(piperidin-1-yl)cyclobutoxy)phenyl)acetamide (BIOS-B-9)

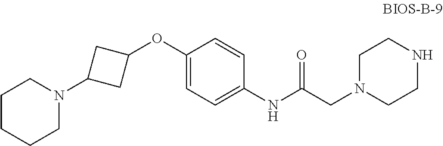 [SMILES: O=C(CN1CCNCC1)Nc1ccc(OC2CC(N3CCCCC3)C2)cc1]

The synthesis route is as follows:

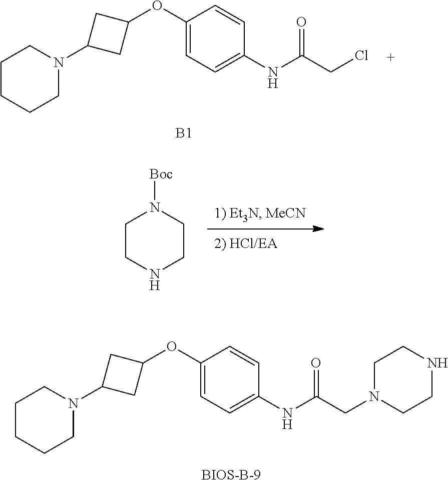 [SMILES: O=C(CCl)Nc1ccc(OC2CC(N3CCCCC3)C2)cc1 ; "B1" ; CC(C)(C)OC(=O)N1CCNCC1 ; O=C(CN1CCNCC1)Nc1ccc(OC2CC(N3CCCCC3)C2)cc1 ; "BIOS-B-9"]

Intermediate B1 (150 mg, 0.465 mmol), 1-Boc-piperazine (260 mg, 1.394 mmol), and 323 μL triethylamine were dissolved in 4 mL acetonitrile and transferred to reaction overnight at 80° C. When the reaction completed, Concentrate the reaction solution, and purified by Thin layer chromatography (PE:EA:TEA=5:4:1), affording 179 mg pale white solid intermediate. 105 mg of the intermediate was reacted with HCl/EA for 1-2 hour, affording 105 mg white solid (the hydrochloride of compound BIOS-B-9), with a two-step yield of 79.89%.

MS: 373.1[M+H]⁺.

¹H NMR (400 MHz, DMSO-d₆) δ 11.27 (s, 1H), 10.72 (s, 1H), 9.82 (s, 2H), 7.56 (d, J=9.0 Hz, 2H), 6.83 (d, J=9.0 Hz, 2H), 4.92˜4.87 (m, 1H), 4.10 (s, 2H), 3.83 (d, J=7.9 Hz, 1H), 3.58˜3.32 (m, 8H), 2.96˜2.89 (m, 2H), 2.73˜2.64 (m, 2H), 2.40˜2.29 (m, 2H), 1.94˜1.65 (m, 5H), 1.40˜1.30 (m, 1H).

### Example 10: Synthesis of 2-(4-methylpiperazin-1-yl)-N-(4-(3-(piperidin-1-yl)cyclobutoxy)phenyl)acetamide (BIOS-B-10)

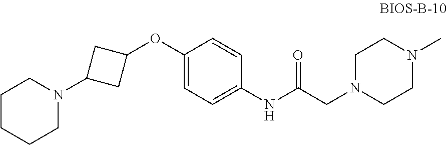 [SMILES: CN1CCN(CC(=O)Nc2ccc(OC3CC(N4CCCCC4)C3)cc2)CC1]

The synthesis route is as follows:

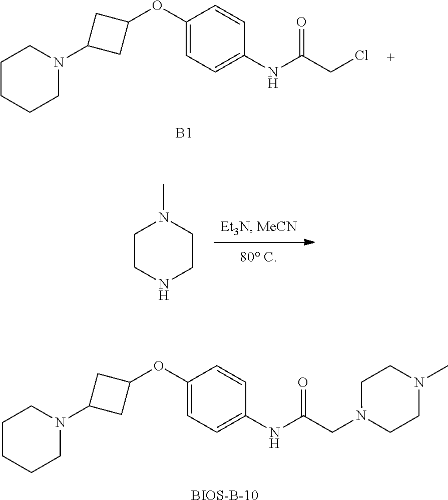

Referred to the synthesis route of compound BIOS-B-3 in Example 3, 4-methylpiperazine from this example was used instead of piperidine from Example 3, and the remaining preparation steps were the same as in Example 3. 107 mg pale yellow solid (compound BIOS-B-10) was obtained with a yield of 89.30%.

MS: 373.1[M+H]⁺.

¹H NMR (400 MHz, DMSO-d₆) δ 9.51 (s, 1H), 7.49 (d, J=9.0 Hz, 2H), 6.75 (d, J=9.0 Hz, 2H), 4.70˜4.65 (m, 1H), 3.06 (s, 2H), 2.86 (d, J=7.8 Hz, 1H), 2.42˜2.20 (m, 14H), 2.18 (s, 3H), 2.13˜2.07 (m, 2H), 1.55˜1.32 (m, 6H).

### Example 11: Synthesis of 2-(3-hydroxypiperidin-1-yl)-N-(4-(3-(piperidin-1-yl)cyclobutoxy)phenyl)acetamide (BIOS-B-11)

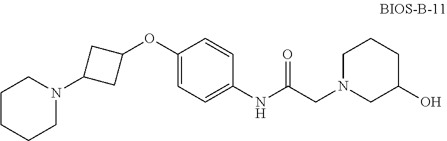 [SMILES: O=C(CN1CCCC(O)C1)Nc1ccc(OC2CC(N3CCCCC3)C2)cc1]

The synthesis route is as follows:

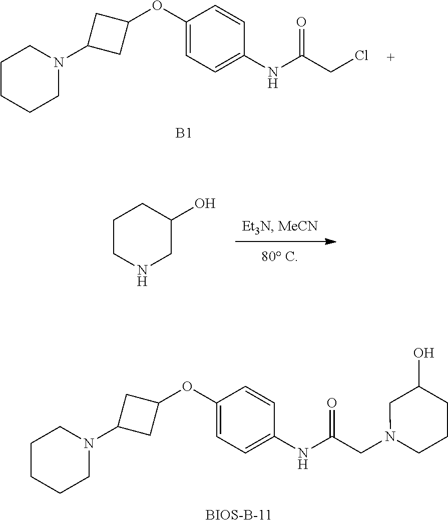 [SMILES: O=C(CCl)Nc1ccc(OC2CC(N3CCCCC3)C2)cc1 ; "B1" ; OC1CCCNC1 ; O=C(CN1CCCC(O)C1)Nc1ccc(OC2CC(N3CCCCC3)C2)cc1 ; "BIOS-B-11"]

Referred to the synthesis route of compound BIOS-B-1 in Example 1, 3-hydroxypyridine ethanol from this example was used instead of morpholine from Example 1, and the remaining preparation steps were the same as in Example 1. 108 mg brown oil (compound BIOS-B-11) was obtained with a yield of 89.90%.

Furthermore, the hydrochloride of compound BIOS-B-8 could be prepared by reacting with HCl/EA for 1-2 hours, which afford 108 mg white solid with a yield of 84.16%.

MS: 388.1[M+H]⁺.

¹H NMR (400 MHz, DMSO-d₆) δ 9.54 (s, 1H), 7.50 (d, J=9.0 Hz, 2H), 6.76 (d, J=9.0 Hz, 2H), 4.82˜4.53 (m, 2H), 3.63˜3.57 (m, 1H), 3.05 (d, J=1.7 Hz, 2H), 2.93˜2.81 (m, 1H), 2.73 (d, J=10.7, 1H), 2.57 (d, J=10.7, 1H), 2.39˜2.02 (m, 10H), 1.72˜1.69 (m, 2H), 1.57˜1.34 (m, 7H), 1.30˜1.09 (m, 1H).

### Example 12: Synthesis of 2-(4-cyclobutylpiperazin-1-yl)-N-(4-(3-(piperidin-1-yl)cyclobutoxy) phenyl)acetamide (BIOS-B-12)

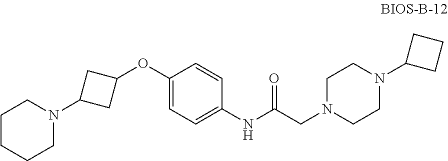 [SMILES: O=C(CN1CCN(C2CCC2)CC1)Nc1ccc(OC2CC(N3CCCCC3)C2)cc1]

The synthesis route is as follows:

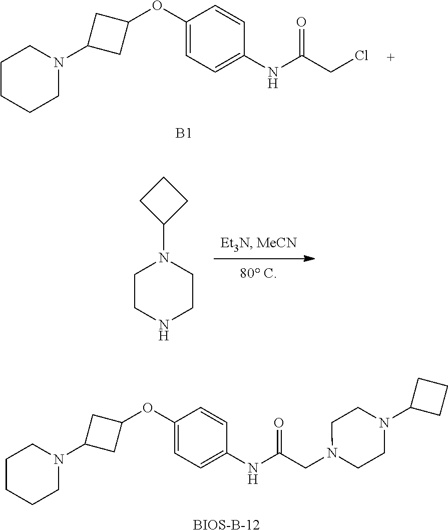 [SMILES: O=C(CCl)Nc1ccc(OC2CC(N3CCCCC3)C2)cc1 ; "B1" ; C1CC(N2CCNCC2)C1 ; O=C(CN1CCN(C2CCC2)CC1)Nc1ccc(OC2CC(N3CCCCC3)C2)cc1 ; "BIOS-B-12"]

Referred to the synthesis route of compound BIOS-B-3 in Example 3, 4-cyclobutylpiperazine from this example was used instead of piperidine from Example 3, and the remaining preparation steps were the same as in Example 3. 50 mg pale yellow solid (compound BIOS-B-12) was obtained with a yield of 37.81%.

MS: 427.3[M+H]⁺.

¹H NMR (400 MHz, DMSO-d₆) δ 9.52 (s, 1H), 7.50 (d, J=9.0 Hz, 2H), 6.75 (d, J=9.0 Hz, 2H), 4.69 (s, 1H), 3.07 (s, 2H), 2.95 (s, 1H), 2.74 (s, 1H), 2.45˜2.04 (m, 12H), 2.02˜1.87 (m, 2H), 1.78 (t, J=9.0 Hz, 2H), 1.70˜1.58 (m, 2H), 1.58˜1.31 (m, 7H), 1.29˜1.20 (m, 1H), 1.16˜0.80 (m, 2H).

### Example 13: Synthesis of 1-(2-morpholinoethyl)-3-(4-(3-(piperidin-1-yl)cyclobutoxy)phenyl)urea (BIOS-B-13)

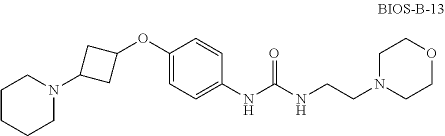 [SMILES: O=C(NCCN1CCOCC1)Nc1ccc(OC2CC(N3CCCCC3)C2)cc1]

The synthesis route is as follows:

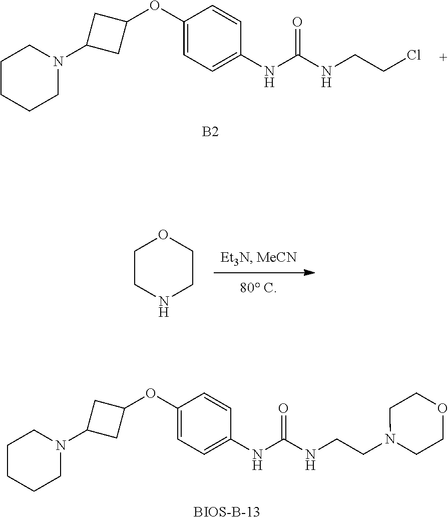 [SMILES: O=C(NCCCl)Nc1ccc(OC2CC(N3CCCCC3)C2)cc1 ; "B2" ; C1COCCN1 ; O=C(NCCN1CCOCC1)Nc1ccc(OC2CC(N3CCCCC3)C2)cc1 ; "BIOS-B-13"]

Intermediate B2 (100 mg, 0.284 mmol), morpholine (75 μL, 0.853 mmol), and 200 μL triethylamine were dissolved in 4 mL acetonitrile and transferred to reaction overnight at 80° C. When the reaction completed, concentrate the reaction solution, and purified by Thin layer chromatography (EA:TEA=4:1), affording 99 mg white solid (compound BIOS-B-13), with a yield of 86.60%.

MS: 403.1[M+H]⁺.

¹H NMR (400 MHz, DMSO-d₆) δ 8.40 (s, 1H), 7.25 (d, J=9.0 Hz, 2H), 6.68 (d, J=9.0 Hz, 2H), 5.95 (t, J=5.4 Hz, 1H), 4.68˜4.59 (m, 1H), 3.59 (t, J=4.7 Hz, 4H), 3.25˜3.15 (m, 2H), 2.95˜2.83 (m, 1H), 2.42˜2.21 (m, 12H), 2.15˜2.05 (m, 2H), 1.55˜1.32 (m, 6H).

### Example 14: Synthesis of (R)-1-(2-(2-methylpyrrolidin-1-yl)ethyl)-3-(4-(3-(piperidin-1-yl)cyclobutoxy)phenyl)urea (BIOS-B-14)

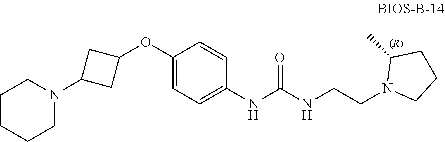

The synthesis route is as follows:

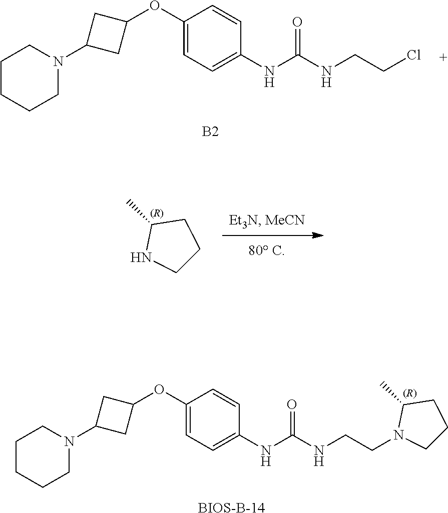

Referred to the synthesis route of compound BIOS-B-13 in Example 13, (R)-2-methylpyrrolidine from this example was used instead of morpholine from Example 13, and the remaining preparation steps were the same as in Example 13. 45 mg white solid (compound BIOS-B-14) was obtained with a yield of 39.56%.

MS: 401.2[M+H]⁺.

¹H NMR (400 MHz, DMSO-d₆) δ 8.42 (s, 1H), 7.25 (d, J=8.4 Hz, 2H), 6.68 (d, J=8.4 Hz, 2H), 5.94 (s, 1H), 4.63 (s, 1H), 3.09 (s, 2H), 2.84 (s, 2H), 2.40˜1.99 (m, 11H), 1.96˜1.82 (m, 1H), 1.66 (t, J=7.8 Hz, 2H), 1.58˜1.20 (m, 8H), 1.04 (d, J=4 Hz, 3H).

### Example 15: Synthesis of 1-(4-(3-(piperidin-1-yl)cyclobutoxy)phenyl)-3-(2-(piperidin-1-yl)ethyl)urea (BIOS-B-15)

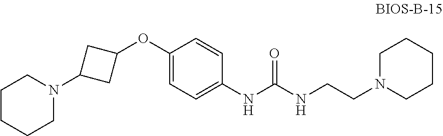 [SMILES: O=C(NCCN1CCCCC1)Nc1ccc(OC2CC(N3CCCCC3)C2)cc1]

The synthesis route is as follows:

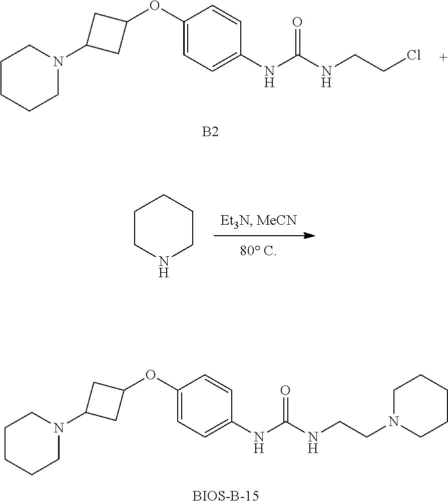 [SMILES: O=C(NCCCl)Nc1ccc(OC2CC(N3CCCCC3)C2)cc1 ; "B2" ; C1CCNCC1 ; O=C(NCCN1CCCCC1)Nc1ccc(OC2CC(N3CCCCC3)C2)cc1 ; "BIOS-B-15"]

Referred to the synthesis route of compound BIOS-B-13 in Example 13, piperidine from this example was used instead of morpholine from Example 13, and the remaining preparation steps were the same as in Example 13. 87 mg white solid (compound BIOS-B-15) was obtained with a yield of 76.48%.

MS: 401.1[M+H]⁺.

¹H NMR (400 MHz, DMSO-d₆) δ 8.42 (s, 1H), 7.25 (d, J=9.0 Hz, 2H), 6.68 (d, J=9.0 Hz, 2H), 5.92 (t, J=5.3 Hz, 1H), 4.68˜4.58 (m, 1H), 3.17 (q, J=6.0 Hz, 2H), 2.86 (s, 1H), 2.46˜2.15 (m, 12H), 2.14˜2.03 (m, 2H), 1.58˜1.30 (m, 12H).

### Example 16: Synthesis of 1-(2-(piperazin-1-yl)ethyl)-3-(4-(3-(piperidin-1-yl)cyclobutoxy)phenyl)urea (BIOS-B-16)

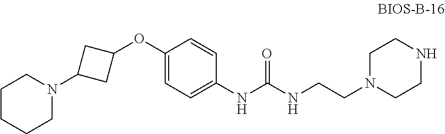 [SMILES: O=C(NCCN1CCNCC1)Nc1ccc(OC2CC(N3CCCCC3)C2)cc1]

The synthesis route is as follows:

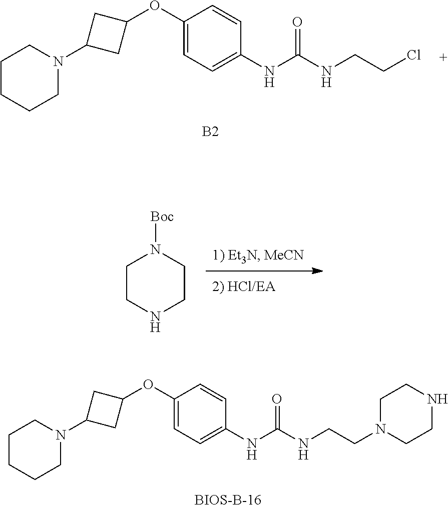 [SMILES: O=C(NCCCl)Nc1ccc(OC2CC(N3CCCCC3)C2)cc1 ; "B2" ; CC(C)(C)OC(=O)N1CCNCC1 ; O=C(NCCN1CCNCC1)Nc1ccc(OC2CC(N3CCCCC3)C2)cc1 ; "BIOS-B-16"]

Intermediate B2 (300 mg, 0.852 mmol), 1-Boc-Piperazine (477 mg, 2.558 mmol), and 600 μL triethylamine were dissolved in 4 mL acetonitrile and transferred to reaction overnight at 80° C. When the reaction completed, concentrate the reaction solution, and purified by Thin layer chromatography (EA:TEA=4:1), affording 346 mg white solid Intermediate. 207 mg of the intermediate was reacted with HCl/EA for 1-2 hours, affording 219 mg white solid (the hydrochloride of compound BIOS-B-16), with a two-step yield of 50.31%.

MS: 402.1[M+H]⁺.

¹H NMR (400 MHz, DMSO-d₆) δ 11.35 (s, 1H), 8.42 (s, 1H), 7.27 (d, J=9.0 Hz, 2H), 6.69 (d, J=9.0 Hz, 2H), 5.95 (t, J=5.5 Hz, 1H), 4.68˜4.59 (m, 1H), 3.22˜3.16 (m, 2H), 2.86 (s, 1H), 2.46˜2.15 (m, 12H), 2.14˜2.03 (m, 2H), 1.58˜1.30 (m, 10H).

### Example 17: Synthesis of 1-(2-(4-methyl-1,4-diazepan-1-yl)ethyl)-3-(4-(3-(piperidin-1-yl)cyclobutoxy)phenyl)urea (BIOS-B-17)

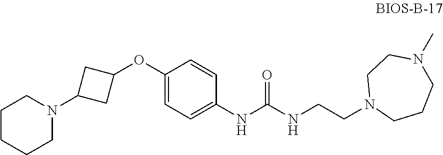 [SMILES: CN1CCCN(CCNC(=O)Nc2ccc(OC3CC(N4CCCCC4)C3)cc2)CC1]

The synthesis route is as follows:

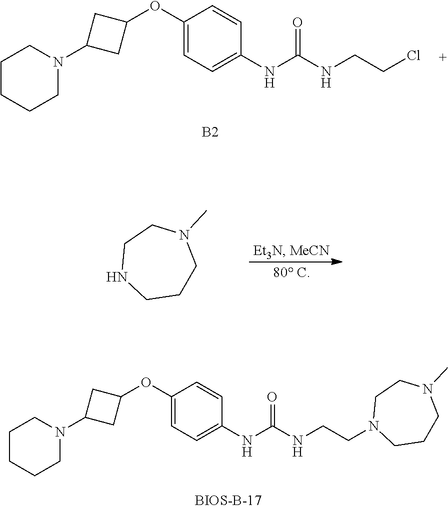

Referred to the synthesis route of compound BIOS-B-13 in Example 13, 1-methyl-1,4-diazepane from this example was used instead of morpholine from Example 13, and the remaining preparation steps were the same as in Example 13. 74 mg yellow solid (compound BIOS-B-17) was obtained with a yield of 60.65%.

MS: 430.2[M+H]⁺.

¹H NMR (400 MHz, DMSO-d₆) δ 8.88 (s, 1H), 7.29 (d, J=9.0 Hz, 2H), 6.69 (d, J=9.0 Hz, 2H), 6.43 (t, J=5.3 Hz, 1H), 4.70˜4.64 (m, 1H), 3.24˜3.21 (m, 4H), 3.18˜3.13 (m, 5H), 3.05˜3.03 (m, 2H), 2.85 (t, J=5.2 Hz, 2H), 2.71 (d, J=3.9 Hz, 5H), 2.62˜2.58 (m, 3H), 2.18 (t, J=9.9 Hz, 2H), 1.99˜1.89 (m, 2H), 1.64˜1.39 (m, 6H), 1.18 (t, J=7.3 Hz, 1H).

### Example 18: Synthesis of 1-(2-(4,4-difluoropiperidin-1-yl)ethyl)-3-(4-(3-(piperidin-1-yl)cyclobutoxy)phenyl)urea (BIOS-B-18)

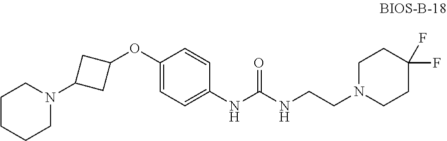 [SMILES: O=C(NCCN1CCC(F)(F)CC1)Nc1ccc(OC2CC(N3CCCCC3)C2)cc1]

The synthesis route is as follows:

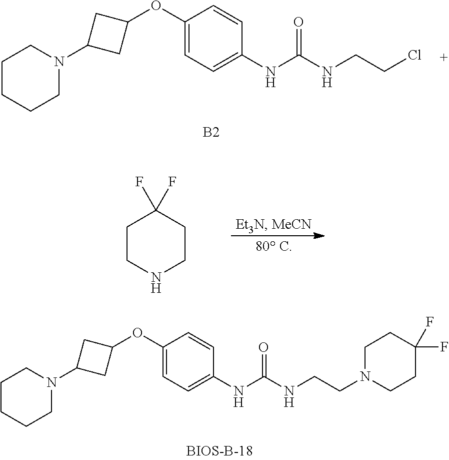 [SMILES: O=C(NCCCl)Nc1ccc(OC2CC(N3CCCCC3)C2)cc1 ; "B2" ; FC1(F)CCNCC1 ; O=C(NCCN1CCC(F)(F)CC1)Nc1ccc(OC2CC(N3CCCCC3)C2)cc1]

Referred to the synthesis route of compound BIOS-B-13 in Example 13, 4,4-difluoropiperidine from this example was used instead of morpholine from Example 13, and the remaining preparation steps were the same as in Example 13. 97 mg yellow solid (compound BIOS-B-18) was obtained with a yield of 78.24%.

MS: 437.1[M+H]⁺.

¹H NMR (400 MHz, DMSO-d₆) δ 8.36 (s, 1H), 7.25 (d, J=9.0 Hz, 2H), 6.68 (d, J=9.0 Hz, 2H), 5.96 (t, J=5.5 Hz, 1H), 4.68˜4.58 (m, 1H), 3.18 (q, J=6.1 Hz, 2H), 2.87 (s, 1H), 2.58˜2.51 (m, 4H), 2.45 (t, J=6.4 Hz, 2H), 2.37˜2.16 (m, 6H), 2.14˜2.03 (m, 2H), 2.02˜1.88 (m, 4H), 1.56˜1.33 (m, 6H).

### Example 19: Synthesis of 1-(2-(4-hydroxypiperidin-1-yl)ethyl)-3-(4-(3-(piperidin-1-yl)cyclobutoxy)phenyl)urea (BIOS-B-19)

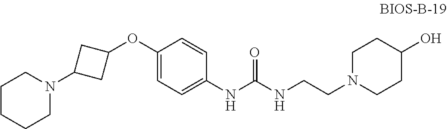 [SMILES: O=C(NCCN1CCC(O)CC1)Nc1ccc(OC2CC(N3CCCCC3)C2)cc1]

The synthesis route is as follows:

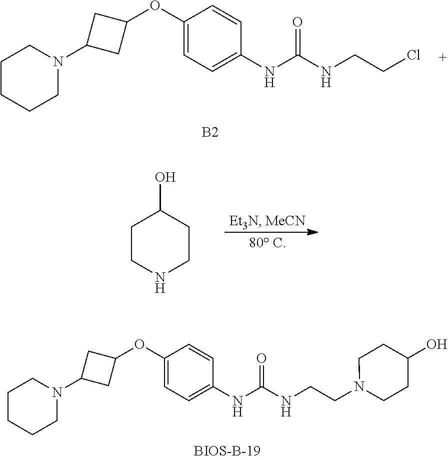 [SMILES: O=C(NCCCl)Nc1ccc(OC2CC(N3CCCCC3)C2)cc1 ; "B2" ; OC1CCNCC1 ; O=C(NCCN1CCC(O)CC1)Nc1ccc(OC2CC(N3CCCCC3)C2)cc1]

Referred to the synthesis route of compound BIOS-B-13 in Example 13, 4-hydroxypyridine from this example was used instead of morpholine from Example 13, and the remaining preparation steps were the same as in Example 13. 97 mg pink solid (compound BIOS-B-19) was obtained with a yield of 81.99%.

MS: 437.1[M+H]⁺.

¹H NMR (400 MHz, DMSO-d₆) δ 8.42 (s, 1H), 7.25 (d, J=8.4 Hz, 2H), 6.68 (d, J=8.4 Hz, 2H), 5.93 (t, J=5.4 Hz, 1H), 4.69˜4.59 (m, 1H), 4.57 (s, 1H), 3.45 (s, 1H), 3.16 (q, J=6.0 Hz, 2H), 2.93˜2.85 (m, 1H), 2.81˜2.64 (m, 2H), 2.42˜2.16 (m, 8H), 2.14˜1.94 (m, 4H), 1.81˜1.62 (m, 2H), 1.56˜1.29 (m, 8H).

### Example 20: Synthesis of 1-(4-(3-(piperidin-1-yl)cyclobutoxy)phenyl)-3-(2-thiomorpholinoethyl)urea (BIOS-B-20)

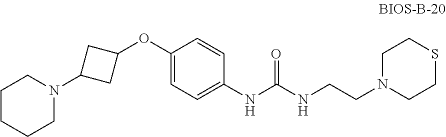 [SMILES: O=C(NCCN1CCSCC1)Nc1ccc(OC2CC(N3CCCCC3)C2)cc1]

The synthesis route is as follows:

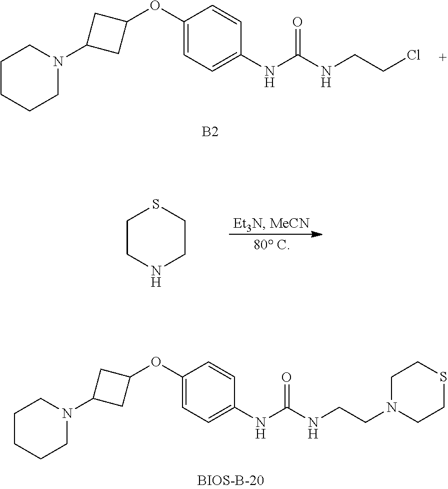 [SMILES: O=C(NCCCl)Nc1ccc(OC2CC(N3CCCCC3)C2)cc1 ; "B2" ; C1CSCCN1 ; O=C(NCCN1CCSCC1)Nc1ccc(OC2CC(N3CCCCC3)C2)cc1]

Referred to the synthesis route of compound BIOS-B-13 in Example 13, thiomorpholine from this example was used instead of morpholine from Example 13, and the remaining preparation steps were the same as in Example 13. 103 mg yellow solid (compound BIOS-B-20) was obtained with a yield of 86.64%.

MS: 419.1[M+H]⁺.

¹H NMR (400 MHz, DMSO-d₆) δ 8.36 (s, 1H), 7.25 (d, J=9.0 Hz, 2H), 6.68 (d, J=9.0 Hz, 1H), 5.91 (t, J=5.4 Hz, 1H), 4.68˜4.58 (m, 1H), 3.17 (q, J=6.1 Hz, 2H), 2.92˜2.80 (m, 1H), 2.72˜2.57 (m, 8H), 2.40 (t, J=6.4 Hz, 2H), 2.35˜2.16 (m, 6H), 2.14˜2.03 (m, 2H), 1.54˜1.32 (m, 6H).

### Example 21: Synthesis of 1-(2-(2-(2-hydroxyethyl)piperidin-1-yl)ethyl)-3-(4-(3-(piperidin-1-yl)cyclobutoxy)phenyl)urea (BIOS-B-21)

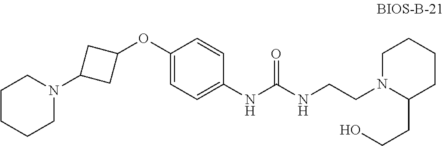 [SMILES: O=C(NCCN1CCCCC1CCO)Nc1ccc(OC2CC(N3CCCCC3)C2)cc1]

The synthesis route is as follows:

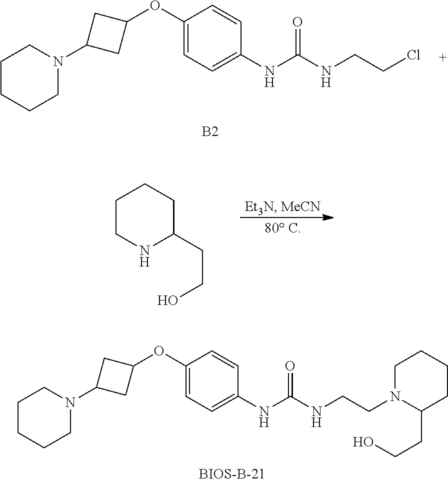 [SMILES: O=C(NCCCl)Nc1ccc(OC2CC(N3CCCCC3)C2)cc1 ; "B2" ; OCCC1CCCCN1 ; O=C(NCCN1CCCCC1CCO)Nc1ccc(OC2CC(N3CCCCC3)C2)cc1]

Referred to the synthesis route of compound BIOS-B-13 in Example 13, 2-piperidine ethanol from this example was used instead of morpholine from Example 13, and the remaining preparation steps were the same as in Example 13. 74 mg yellow solid (compound BIOS-B-21) was obtained with a yield of 58.60%.

MS: 445.2[M+H]⁺.

¹H NMR (400 MHz, DMSO-d₆) δ 8.45 (s, 1H), 7.26 (d, J=8.9 Hz, 2H), 6.68 (d, J=8.9 Hz, 2H), 5.92 (t, J=5.4 Hz, 1H), 4.68˜4.58 (m, 1H), 4.32 (s, 1H), 3.25˜3.12 (m, 4H), 2.86 (s, 1H), 2.46˜2.15 (m, 11H), 2.14˜2.03 (m, 2H), 1.58˜1.30 (m, 14H).

### Example 22: Synthesis of 1-(2-(4-methylpiperazin-1-yl)ethyl)-3-(4-(3-(piperidin-1-yl)cyclobutoxy)phenyl)urea (BIOS-B-22)

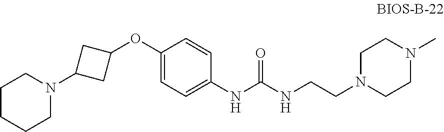 [SMILES: CN1CCN(CCNC(=O)Nc2ccc(OC3CC(N4CCCCC4)C3)cc2)CC1]

The synthesis route is as follows:

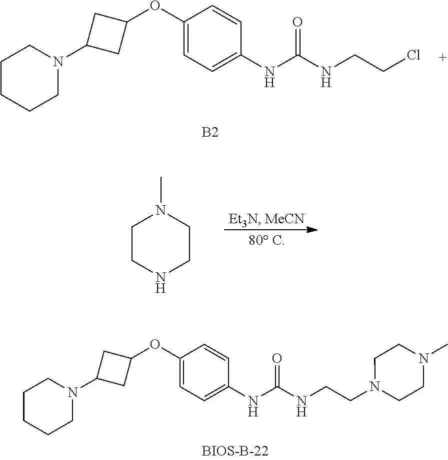

Referred to the synthesis route of compound BIOS-B-13 in Example 13, 4-methylpiperazine from this example was used instead of morpholine from Example 13, and the remaining preparation steps were the same as in Example 13. 101 mg yellow solid (compound BIOS-B-22) was obtained with a yield of 85.58%.

MS: 416.2[M+H]⁺.

¹H NMR (400 MHz, DMSO-d₆) δ 8.45 (s, 1H), 7.27 (d, J=8.8 Hz, 2H), 6.70 (d, J=8.8 Hz, 2H), 5.97 (s, 1H), 4.68 (s, 1H), 3.22˜3.16 (m, 3H), 3.05 (s, 2H), 2.67 (s, 1H), 2.44˜2.04 (m, 14H), 1.68˜1.32 (m, 7H), 1.29˜1.08 (m, 3H).

### Example 23: Synthesis of 1-(2-(3-hydroxypiperidin-1-yl)ethyl)-3-(4-(3-(piperidin-1-yl)cyclobutoxy)phenyl)urea (BIOS-B-23)

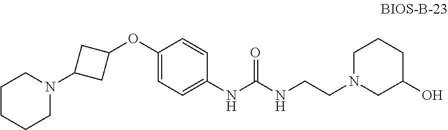 [SMILES: O=C(NCCN1CCCC(O)C1)Nc1ccc(OC2CC(N3CCCCC3)C2)cc1]

The synthesis route is as follows:

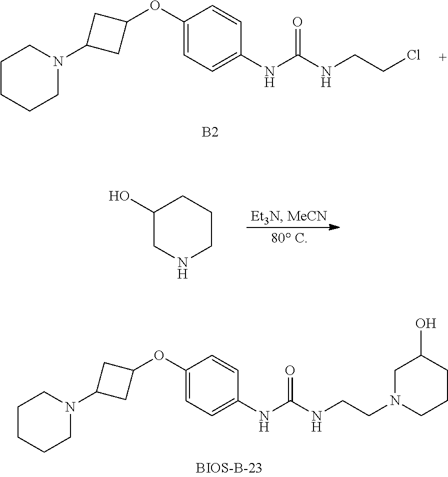 [SMILES: O=C(NCCCl)Nc1ccc(OC2CC(N3CCCCC3)C2)cc1 ; "B2" ; OC1CCCNC1 ; O=C(NCCN1CCCC(O)C1)Nc1ccc(OC2CC(N3CCCCC3)C2)cc1]

Referred to the synthesis route of compound BIOS-B-13 in Example 13, 3-hydroxypyridine from this example was used instead of morpholine from Example 13, and the remaining preparation steps were the same as in Example 13. 103 mg yellow solid (compound BIOS-B-23) was obtained with a yield of 87.06%.

MS: 417.1[M+H]⁺.

¹H NMR (400 MHz, DMSO-d₆) δ 8.41 (s, 1H), 7.26 (d, J=9.2 Hz, 2H), 6.68 (d, J=9.2 Hz, 2H), 5.94 (t, J=5.3 Hz, 1H), 4.68˜4.61 (m, 1H), 4.61˜4.53 (m, 1H), 3.53˜3.46 (m, 1H), 3.18˜3.12 (m, 2H), 2.91˜2.78 (m, 2H), 2.77˜2.61 (m, 2H), 2.43˜2.17 (m, 8H), 2.14˜2.03 (m, 2H), 1.94˜1.71 (m, 3H), 1.67˜1.57 (m, 1H), 1.54˜1.35 (m, 7H).

### Example 24: Synthesis of 1-(2-(4-cyclobutylpiperazin-1-yl)ethyl)-3-(4-(3-(piperidin-1-yl)cyclobutoxy)phenyl)urea (BIOS-B-24)

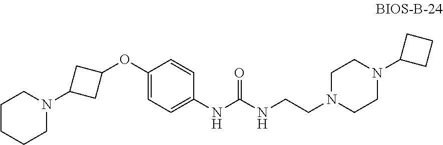 [SMILES: O=C(NCCN1CCN(C2CCC2)CC1)Nc1ccc(OC2CC(N3CCCCC3)C2)cc1]

The synthesis route is as follows:

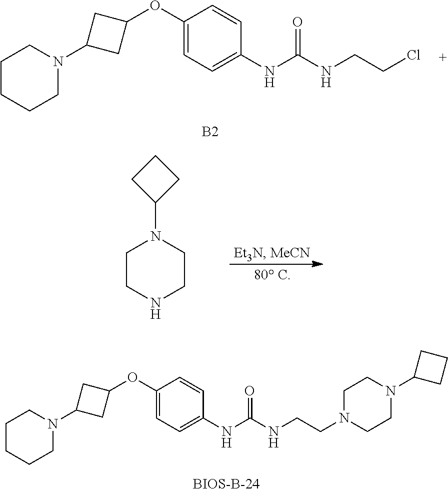 [SMILES: O=C(NCCCl)Nc1ccc(OC2CC(N3CCCCC3)C2)cc1 ; "B2" ; C1CC(N2CCNCC2)C1 ; O=C(NCCN1CCN(C2CCC2)CC1)Nc1ccc(OC2CC(N3CCCCC3)C2)cc1]

Referred to the synthesis route of compound BIOS-B-13 in Example 13, 4-cyclobutylpiperazine from this example was used instead of morpholine from Example 13, and the remaining preparation steps were the same as in Example 13. 70 mg yellow solid (compound BIOS-B-24) was obtained with a yield of 54.10%.

MS: 456.1[M+H]⁺.

¹H NMR (400 MHz, DMSO-d₆) δ 8.42 (s, 1H), 7.25 (d, J=9.0 Hz, 2H), 6.67 (d, J=9.0 Hz, 2H), 5.92 (s, 1H), 3.18˜3.14 (m, 2H), 2.88˜2.80 (m, 1H), 2.67 (t, J=7.7 Hz, 1H), 2.45˜2.01 (m, 18H), 1.93 (s, 2H), 1.82˜1.56 (m, 5H), 1.55˜1.32 (m, 6H).

### Example 25: Synthesis of 2-morpholino-N-(4-(3-(piperidin-1-yl)cyclobutoxy)phenyl)ethanethioamide (BIOS-B-25)

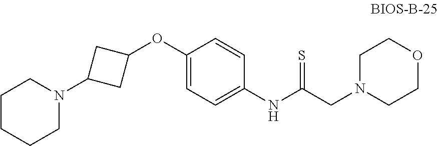 [SMILES: S=C(CN1CCOCC1)Nc1ccc(OC2CC(N3CCCCC3)C2)cc1]

The synthesis route is as follows:

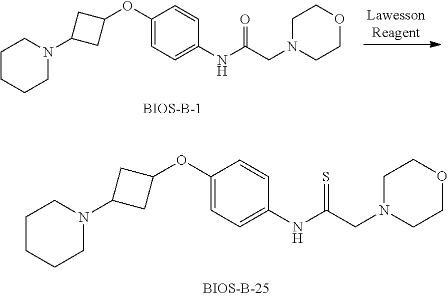 [SMILES: O=C(CN1CCOCC1)Nc1ccc(OC2CC(N3CCCCC3)C2)cc1 ; S=C(CN1CCOCC1)Nc1ccc(OC2CC(N3CCCCC3)C2)cc1]

Compound BIOS-B-1 (100 mg, 0.268 mmol) and Lawesson Reagent (108 mg, 0.268 mmol) were dissolved in 4 mL anhydrous 1,4-dioxane and refluxed for 16 hours. When the reaction is completed, concentrate the reaction solution, and purified by Thin layer chromatography (PE:EA:TEA=5:4:1), affording 12 mg yellow solid (compound BIOS-B-25), with a yield of 11.46%.

MS: 390.1[M+H]⁺.

¹H NMR (400 MHz, DMSO-d₆) δ 9.59 (s, 1H), 7.50 (d, J=9.0 Hz, 2H), 6.75 (d, J=9.0 Hz, 2H), 4.70˜4.65 (m, 1H), 3.70˜3.54 (m, 4H), 3.14 (s, 2H), 2.88˜2.81 (m, 1H), 2.50˜2.46 (m, 4H), 2.37˜2.00 (m, 8H), 1.60˜1.35 (m, 6H).

The synthesis route and preparation steps of compound BIOS-B-1 are described in Example 1 and will not be elaborated here.

### Example 26: Synthesis of (R)-2-(2-methylpyrrolidin-1-yl)-N-(4-(3-(piperidin-1-yl)cyclobutoxy)phenyl)ethanethioamide (BIOS-B-26)

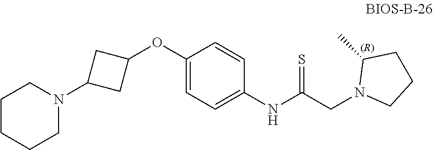

The synthesis route is as follows:

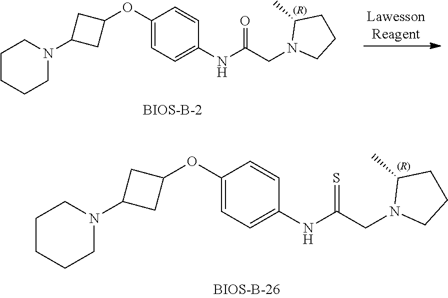

Referred to the synthesis route of compound BIOS-B-25 in Example 25, BIOS-B-2 from this example was used instead of BIOS-B-1 from Example 25, and the remaining preparation steps were the same as in Example 25. 38 mg yellow solid (compound BIOS-B-26) was obtained with a yield of 36.45%.

MS: 388.1[M+H]⁺.

¹H NMR (400 MHz, DMSO-d₆) δ 9.47 (s, 1H), 7.50 (d, J=9.0 Hz, 2H), 6.75 (d, J=9.0 Hz, 2H), 4.69˜4.64 (m, 1H), 3.32 (d, J=15.5 Hz, 1H), 3.11˜3.06 (m, 1H), 2.90 (d, J=15.5 Hz, 1H), 2.88˜2.81 (m, 1H), 2.49˜2.03 (m, 10H), 1.97˜1.87 (m, 1H), 1.81˜1.60 (m, 2H), 1.57˜1.29 (m, 7H), 1.06 (d, J=6.1 Hz, 3H).

The synthesis route and preparation steps of compound BIOS-B-2 are described in Example 2 and will not be elaborated here.

### Example 27: Synthesis of 2-(piperidin-1-yl)-N-(4-(3-(piperidin-1-yl)cyclobutoxy)phenyl)ethanethioamide (BIOS-B-27)

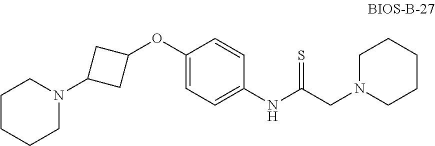 [SMILES: S=C(CN1CCCCC1)Nc1ccc(OC2CC(N3CCCCC3)C2)cc1]

The synthesis route is as follows:

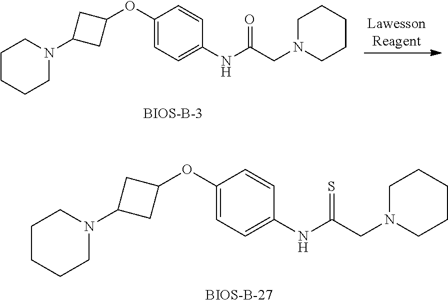 [SMILES: O=C(CN1CCCCC1)Nc1ccc(OC2CC(N3CCCCC3)C2)cc1 ; S=C(CN1CCCCC1)Nc1ccc(OC2CC(N3CCCCC3)C2)cc1]

Referred to the synthesis route of compound BIOS-B-25 in Example 25, BIOS-B-3 from this example was used instead of BIOS-B-1 from Example 25, and the remaining preparation steps were the same as in Example 25. 29 mg yellow solid (compound BIOS-B-27) was obtained with a yield of 27.81%.

MS: 388.1[M+H]⁺.

¹H NMR (400 MHz, DMSO-d₆) δ 9.50 (s, 1H), 7.50 (d, J=9.0 Hz, 2H), 6.75 (d, J=9.0 Hz, 2H), 4.70˜4.65 (m, 1H), 3.10 (s, 2H), 2.95˜2.81 (m, 1H), 2.44 (t, J=5.3 Hz, 4H), 2.38˜2.03 (m, 8H), 1.66˜1.31 (m, 12H).

The synthesis route and preparation steps of compound BIOS-B-3 are described in Example 3 and will not be elaborated here.

### Example 28: Synthesis of 2-(4-methyl-1,4-diazepan-1-yl)-N-(4-(3-(piperidin-1-yl)cyclobutoxy) phenyl)ethanethioamide (BIOS-B-28)

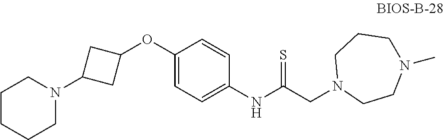 [SMILES: CN1CCCN(CC(=S)Nc2ccc(OC3CC(N4CCCCC4)C3)cc2)CC1]

The synthesis route is as follows:

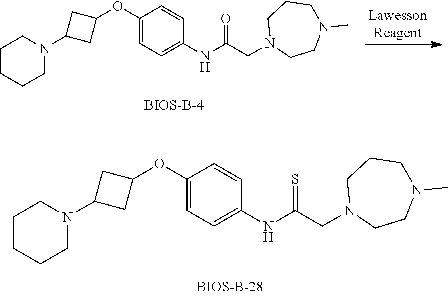 [SMILES: CN1CCCN(CC(=O)Nc2ccc(OC3CC(N4CCCCC4)C3)cc2)CC1 ; CN1CCCN(CC(=S)Nc2ccc(OC3CC(N4CCCCC4)C3)cc2)CC1]

Referred to the synthesis route of compound BIOS-B-25 in Example 25, BIOS-B-4 from this example was used instead of BIOS-B-1 from Example 25, and the remaining preparation steps were the same as in Example 25. 30 mg yellow solid (compound BIOS-B-28) was obtained with a yield of 37.90%.

MS: 417.1[M+H]⁺.

¹H NMR (400 MHz, DMSO-d₆) δ 9.53 (s, 1H), 7.50 (d, J=9.0 Hz, 2H), 6.75 (d, J=9.0 Hz, 2H), 4.70˜4.64 (m, 1H), 3.28 (s, 2H), 2.87˜2.83 (m, 1H), 2.80˜2.71 (m, 4H), 2.64˜2.56 (m, 4H), 2.38˜2.14 (m, 9H), 2.12˜2.06 (m, 2H), 1.83˜1.71 (m, 2H), 1.56˜1.32 (m, 6H).

The synthesis route and preparation steps of compound BIOS-B-4 are described in Example 4 and will not be elaborated here.

### Example 29: Synthesis of 2-(diethylamino)-N-(4-(3-(piperidin-1-yl)cyclobutoxy)phenyl)acetamide (BIOS-B-29)

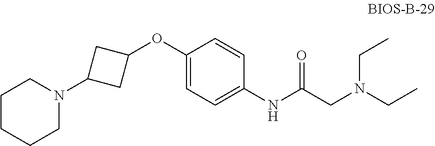 [SMILES: CCN(CC)CC(=O)Nc1ccc(OC2CC(N3CCCCC3)C2)cc1]

The synthesis route is as follows:

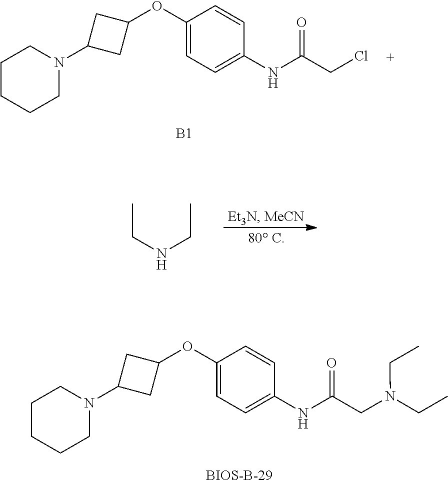

Referred to the synthesis route of compound BIOS-B-1 in Example 1, diethylamine from this example was used instead of morpholine from Example 1, and the remaining preparation steps were the same as in Example 1. 69 mg pale yellow (compound BIOS-B-29) was obtained with a yield of 61.95%.

MS: 360.1[M+H]⁺.

¹H NMR (400 MHz, DMSO-d₆) δ 9.49 (s, 1H), 7.50 (d, J=8.8 Hz, 2H), 6.75 (d, J=8.8 Hz, 2H), 4.70˜4.65 (m, 1H), 3.18 (s, 2H), 2.95˜2.81 (m, 1H), 2.44 (t, J=5.5 Hz, 4H), 2.29˜2.08 (m, 8H), 1.66˜1.26 (m, 12H).

### Example 30: Synthesis of 2-(dimethylamino)-N-(4-(3-(piperidin-1-yl)cyclobutoxy)phenyl)acetamide (BIOS-B-30)

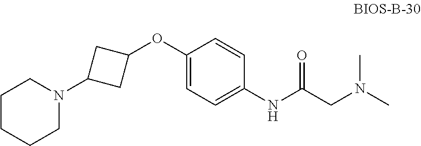 [SMILES: CN(C)CC(=O)Nc1ccc(OC2CC(N3CCCCC3)C2)cc1]

The synthesis route is as follows:

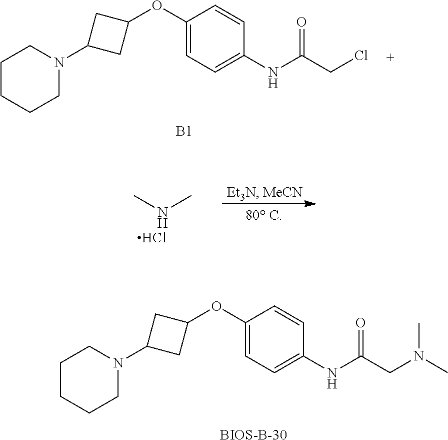

Referred to the synthesis route of compound BIOS-B-1 in Example 1, dimethylamine hydrochloride from this example was used instead of morpholine from Example 1, and the remaining preparation steps were the same as in Example 1. 51 mg pale yellow (compound BIOS-B-30) was obtained with a yield of 49.67%.

MS: 332.1[M+H]⁺.

¹H NMR (400 MHz, DMSO-d₆) δ 9.56 (s, 1H), 7.51 (d, J=8.9 Hz, 2H), 6.77 (d, J=8.9 Hz, 2H), 4.70˜4.65 (m, 1H), 3.12 (s, 2H), 2.95˜2.82 (m, 1H), 2.45 (t, J=5.6 Hz, 4H), 2.29˜2.08 (m, 10H), 1.66˜1.26 (m, 6H).

### Example 31: Synthesis of 2-(2-(dimethylamino)ethoxy)-N-(4-(3-(piperidin-1-yl)cyclobutoxy) phenyl)acetamide (BIOS-B-31)

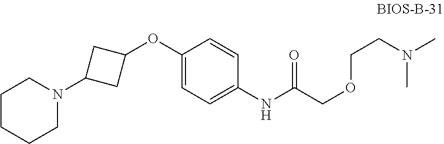 [SMILES: CN(C)CCOCC(=O)Nc1ccc(OC2CC(N3CCCCC3)C2)cc1]

The synthesis route is as follows:

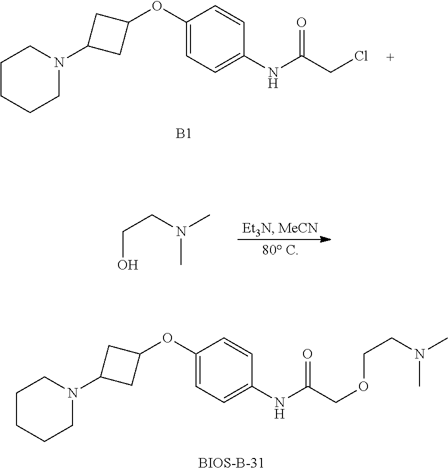

Referred to the synthesis route of compound BIOS-B-1 in Example 1, N,N-Dimethylethanolamine from this example was used instead of morpholine from Example 1, and the remaining preparation steps were the same as in Example 1.67 mg pale yellow (compound BIOS-B-31) was obtained with a yield of 57.58%.

MS: 376.1[M+H]⁺.

¹H NMR (400 MHz, DMSO-d₆) δ 9.26 (s, 1H), 7.53 (d, J=8.6 Hz, 2H), 6.85 (d, J=8.6 Hz, 2H), 4.65 (t, J=9.9 Hz, 1H), 4.16 (s, 2H), 3.69 (t, J=6.7 Hz, 2H), 3.02 (t, J=6.3 Hz, 1H), 2.81˜2.69 (m, 5H), 2.58˜2.43 (m, 3H), 2.36˜2.30 (m, 3H), 2.18˜2.02 (m, 3H), 1.57˜1.38 (m, 8H).

### Example 32: The Antagonistic of Compounds Target to Histamine H3 Receptor

The FLPPR assay (NIH Assay Guidance Manual: HTS Assay Validation-Section 4.3. Analysis (Potency), htpps://www.ncbi.nlm.nih.gov/books/NBK83783) was performed using SUVN-G3031 as a positive control, and 21 target compounds were tested for the antagonistic activity target to histamine H3 receptor. The results are shown in Table 1.

TABLE 1
The antagonistic of compounds target to Histamine H3 receptor

| | % Inh-AVERAGE @ | % Inh-AVERAGE @ |
|---|---|---|
| Compound ID | 50 nM | 5 nM |
| Positive control | 85.82 | 57.76 |
| SUVN-G3031 | | |
| BIOS-B-1 | 100.18 | 91.74 |
| BIOS-B-2 | 100.21 | 100.12 |
| BIOS-B-3 | 99.37 | 95.56 |
| BIOS-B-4 | 100.21 | 101.36 |
| BIOS-B-5 | 99.89 | 98.88 |
| BIOS-B-6 | 99.75 | 102.75 |
| BIOS-B-7 | 99.51 | 88.27 |
| BIOS-B-8 | 99.46 | 101.42 |
| BIOS-B-9 | 99.60 | 100.64 |
| BIOS-B-10 | 97.46 | 99.46 |
| BIOS-B-11 | 99.43 | 97.00 |
| BIOS-B-12 | 98.81 | 99.08 |
| BIOS-B-13 | 99.76 | 99.56 |
| BIOS-B-14 | 100.58 | 100.07 |
| BIOS-B-15 | 99.95 | 98.97 |
| BIOS-B-17 | 99.40 | 100.54 |
| BIOS-B-18 | 99.87 | 99.72 |
| BIOS-B-19 | 100.86 | 100.62 |
| BIOS-B-20 | 100.51 | 100.27 |
| BIOS-B-22 | 99.52 | 101.10 |
| BIOS-B-23 | 100.31 | 100.47 |

As shown in Table 1, some compounds showed strong histamine H3 receptor antagonist activity, and most of them were more active than the positive control at 5 nM.

### Example 33: The hERG Inhibition of Compounds

In cardiomyocytes, inhibition of the delayed rectifier potassium current (IKr) channel protein encoded by the human Ether-a-go-go Related Gene (hERG) is the most important mechanism by which drugs lead to prolongation of the QT interval. hERG is characterized by a specific molecular structure, which allows it to be inhibited by different structures, leading to severe arrhythmias. Statistically, 25-40% of lead compounds show varying degrees of hERG-related toxicity. Therefore, early evaluation of the effects of compounds on hERG is crucial in the drug development process.

The inhibition rate of compounds on hERG channels was examined by manual membrane clamp technique on HEK293 cell line which is stably expressing hERG ion channels. 100 nM of hERG inhibitor Cisapride was used as positive control.

The results are shown in Table 2. The inhibition rate of compounds, such as BIOS-B-17˜BIOS-B-23, BIOS-B-8˜BIOS-B-15, BIOS-B-6, and BIOS-B-4 (at 10 μM), showed less than 5% on hERG channel, which indicated that the tested compounds have a good security.

TABLE 2
The inhibition rate of compounds on hERG channel

Average inhibition rate (%)

| Compound ID | 10 μM | 100 nM |
|---|---|---|
| Cisapride | — | 86.6 |
| BIOS-B-1 | 8.54 | — |
| BIOS-B-4 | 0.46 | — |
| BIOS-B-6 | −0.49 | — |
| BIOS-B-8 | 4.82 | — |
| BIOS-B-9 | −1.03 | — |
| BIOS-B-10 | −1.26 | — |
| BIOS-B-11 | 2.96 | — |
| BIOS-B-12 | 2.06 | — |
| BIOS-B-13 | −1.47 | — |
| BIOS-B-14 | 0.17 | — |
| BIOS-B-15 | −0.12 | — |
| BIOS-B-17 | 1.52 | — |
| BIOS-B-18 | −0.65 | — |
| BIOS-B-19 | −0.09 | — |
| BIOS-B-20 | −0.66 | — |
| BIOS-B-22 | −2.43 | — |
| BIOS-B-23 | 0.37 | — |

Note:
“—” refers not measured.

### Example 34: The Analgesic Effects of Compounds BIOS-B-12 and BIOS-B-14

Exposing the sciatic nerve on male SD rats' (180-200 g) right hind leg, in the anterior segment where the sciatic nerve is about to bifurcate, use sterile chromic gut thread (No. 4, 0.15 mm in diameter) to ligate 4 loops loosely, with each loop 1-2 mm apart, and suture the muscle and skin to establish the chronic constriction injury (CCI) model. In the sham-operated group, only the sciatic nerve was exposed without ligation, and the muscle and skin were sutured. The mechanical withdrawal threshold was detected by electronic analgesia meter (IITC-2391) on the 7^{th} day for 2 consecutive days.

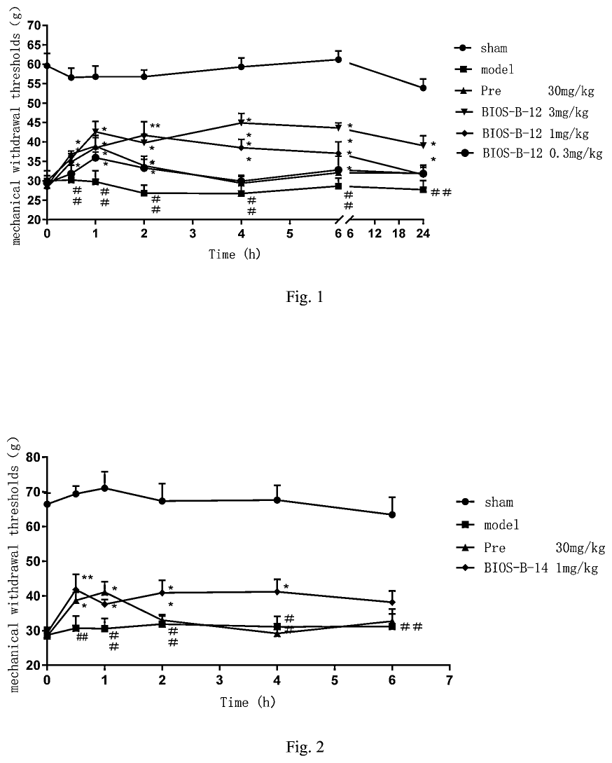

The CCI model rats with stabilized mechanical withdrawal thresholds were randomly divided into five groups, named model group (model), pregabalin group (Pre, 30 mg/kg), BIOS-B-12 compound group (3 mg/kg), BIOS-B-12 compound group (1 mg/kg), and BIOS-B-12 compound group (0.3 mg/kg), and were administered at 1 mL/100 g by gavage, and the same column of saline was gavaged in Sham an model group. The basic value was determined before drug administration, and the mechanical withdrawal thresholds were detected at 0.5 h, 1 h, 2 h, 4 h, 6 h, and 24 h after a single drug administration, and the p value was calculated by ANOVA with T-test. The results are shown in FIG. 1.

The results showed that after the injection of BIOS-B-12 at three doses of 3, 1, and 0.3 mg/kg in a single dose, respectively, a dose-dependently increasing of the mechanical withdrawal thresholds in the CCI model rats was observed. BIOS-B-12 (3 mg/kg) at the time point of 0.5 h to 24 h after the administration of the compound were significantly different from those of the model group (P<0.01). BIOS-B-12 (1 mg/kg) at the time point of 0.5 h to 6 h after the administration of the compound were significantly different from those of the model group (P<0.01). BIOS-B-12 (0.3 mg/kg) at the time point of 1 h to 2 h after the administration of the compound were significantly different from those of the model group (P<0.05). Pregabalin (30 mg/kg) only increased the mechanical withdrawal thresholds of CCI rats from 0.5 h to 2 h after administration, which was significantly different from that of the model group, indicating that the compound BIOS-B-12 acted for a longer period of analgesia time, and the level of analgesia was superior to that of pregabalin.

The CCI model rats with stabilized mechanical withdrawal thresholds were randomly divided into three groups, named model group (model), pregabalin group (Pre, 30 mg/kg), and BIOS-B-14 compound group (1 mg/kg), and were administered at 1 mL/100 g by gavage, and the same column of saline was gavaged in Sham and model group. The basic value was determined before drug administration, and the mechanical withdrawal thresholds were detected at 0.5 h, 1 h, 2 h, 4 h, and 6 h after a single drug administration, and the p value was calculated by ANOVA with T-test. The results are shown in FIG. 2.

The results showed that for the CCI neuropathic pain model in SD rats, the compound BIOS-B-14 (1 mg/kg) significantly increased the mechanical withdrawal thresholds, and the mechanical withdrawal thresholds at the time points from 0.5 h to 5 h after administration of the compound were significantly different from those of the model group (P<0.05). Pregabalin (30 mg/kg) only increased the mechanical withdrawal thresholds of CCI rats from 0.5 h to 1 h after administration, which was significantly different from that of the model group, indicating that the compound BIOS-B-14 acted for a longer period of analgesia time than that of pregabalin.

### Example 35: Antipruritic Ability of Compounds BIOS-B-12 and BIOS-B-18

The C57BL/6J male mice, with 6-8 weeks old, were randomly divided into model group (Model), BIOS-B-12 compound group (10 mg/kg), BIOS-B-18 compound group (10 mg/kg) and dexamethasone group (Dex, 3 mg/kg), with 7 animals in each group. The mice were injected subcutaneously with 500 μg/50 μL histamine at the back of the neck after the administration of dexamethasone for 1 h, BIOS-B-12 for 4 h, and BIOS-B-18 for 1 h, respectively, and immediately observed for 30 min. The number of scratched was recorded and the inhibition rate was calculated as (Model-Compound)/Model×100%.

TABLE 3
Antipruritic ability of compounds BIOS-B-12 and BIOS-B-18

| Compound ID | Inhibition Rate (%) |
|---|---|
| BIOS-B-12 | 36.6 |
| BIOS-B-18 | 55.0 |
| Dex | 29.3 |

As shown in Table 3, BIOS-B-12 (10 mg/kg) and BIOS-B-18 (10 mg/kg) reduced the number of histamine-induced scratched in mice and were more effective than the positive control decamethasone.

1. A histamine-H3-receptor antagonist having the structure of formula (I) or a pharmaceutically acceptable salt thereof: 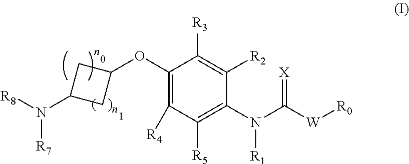
wherein:
W is selected from —(CH₂)ₘ or —NR₆(CH₂)ₘ—;
X is selected from O or S;
n₀ and n₁ are each independently selected from 1, 2 or 3;
m is selected from 0, 1, 2, 3, 4, 5 or 6;
R₀ is selected from —NR′R″, —O(CH₂)ₘNR′R″, substituted or unsubstituted 5-8 membered heterocyclyl, substituted or unsubstituted 5-8 membered heteroaryl; wherein the heterocyclyl or heteroaryl is each independently and optionally substituted with one or more substituents each independently selected from C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, C₁₋₆ alkylsulfoxide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-6 membered cycloalkyl, 3-6 membered heterocyclyl, aryl or heteroaryl;
R′ and R″ are each independently selected from hydrogen, C₁₋₆ alkyl, C₁₋₆ alkoxy, halogen, amino, hydroxy, carboxyl, carbonyl, amide, cyano, C₁₋₆ haloalkyl, C₁₋₆ alkylhydroxy, C₁₋₆ alkylamino, C₁₋₆ alkylamide, C₁₋₆ haloalkoxy, C₁₋₆ alkoxyhydroxy, C₁₋₆ alkoxyamino, or C₁₋₆ alkoxyamide, and R′ and R″ are not hydrogen at the same time;
R₁ and R₆ are each independently selected from hydrogen, C₁₋₆ alkyl, —C(O)-alkyl or —S(O)₂-alkyl;
R₂, R₃, R₄, and R₅ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, or C₁₋₆ alkylsulfoxide;
or, R₂, R₃ and the two carbon atoms on the benzene ring to which R₂ and R₃ are connected respectively, form a substituted or unsubstituted benzobicyclic structure, wherein the benzobicyclic structure may be a benzoheterocyclic ring, but is not limited thereto;
or, R₄, R₅ and the two carbon atoms on the benzene ring to which R₄ and R₅ are connected respectively, form a substituted or unsubstituted benzobicyclic structure; wherein the benzobicyclic structure may be a benzoheterocyclic ring, but is not limited thereto;
R₇ and R₈ are each independently selected from hydrogen, halogen, C₁₋₆ alkyl, C₂₋₆ alkenyl, C₂₋₆ alkynyl, amino, cyano, amide, sulfone, sulfoxide, C₁₋₆ haloalkyl, C₁₋₆ alkylhydroxy, C₁₋₆ alkylamino, C₁₋₆ alkylamide, C₁₋₆ haloalkoxy, C₁₋₆ alkoxyhydroxy, C₁₋₆ alkoxyamino, C₁₋₆ alkoxyamide, C₁₋₆ alkoxysulfone or C₁₋₆ alkoxysulfoxide;
or, R₇, R₈ and the nitrogen atom to which they are both connected, form a ring, and the ring is optionally substituted with one or more substituents each independently selected from hydrogen, halogen, C₁₋₆ alkyl, C₁₋₆ alkoxy, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-8 membered heterocyclyl, or 3-8 membered heteroaryl;
the heterocyclyl, heteroaryl and benzoheterocyclic groups each contain at least one heteroatom selected from N, O or S.

2. The histamine-H3-receptor antagonist of claim 1, having the structure of formula (II) or a pharmaceutically acceptable salt thereof: 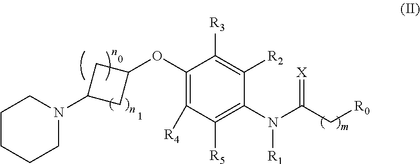
wherein:
X is selected from O or S;
n₀ and n₁ are each independently selected from 1, 2 or 3;
m is selected from 0, 1, 2, 3, 4, 5 or 6;
R₀ is selected from —NR′R″, —O(CH₂)ₘNR′R″, substituted or unsubstituted 5-8 membered heterocyclyl, substituted or unsubstituted 5-8 membered heteroaryl; wherein the heterocyclyl or heteroaryl is each independently and optionally substituted with one or more substituents each independently selected from C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, C₁₋₆ alkylsulfoxide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-6 membered cycloalkyl, 3-6 membered heterocyclyl, aryl or heteroaryl;
R′ and R″ are each independently selected from hydrogen, C₁₋₆ alkyl, C₁₋₆ alkoxy, halogen, amino, hydroxy, carboxyl, carbonyl, amide, cyano, C₁₋₆ haloalkyl, C₁₋₆ alkylhydroxy, C₁₋₆ alkylamino, C₁₋₆ alkylamide, C₁₋₆ haloalkoxy, C₁₋₆ alkoxyhydroxy, C₁₋₆ alkoxyamino, or C₁₋₆ alkoxyamide, and R′ and R″ are not hydrogen at the same time;
R₁ is selected from hydrogen, C₁₋₆ alkyl, —C(O)-alkyl or —S(O)₂-alkyl;
R₂, R₃, R₄, and R₅ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, or C₁₋₆ alkylsulfoxide;
or, R₂, R₃ and the two carbon atoms on the benzene ring to which R₂, R₃ are connected respectively, form a substituted or unsubstituted benzobicyclic structure, wherein the benzobicyclic structure may be a benzoheterocyclic ring, but is not limited thereto;
or, R₄, R₅ and the two carbon atoms on the benzene ring to which R₄, R₅ are connected respectively, form a substituted or unsubstituted benzobicyclic structure; wherein the benzobicyclic structure may be a benzoheterocyclic ring, but is not limited thereto;
each of the heterocyclyl, heteroaryl and benzoheterocyclic group contains at least one heteroatom selected from N, O or S.

3. The histamine-H3-receptor antagonist of claim 2, having the structure of formula (II-1) or a pharmaceutically acceptable salt thereof: 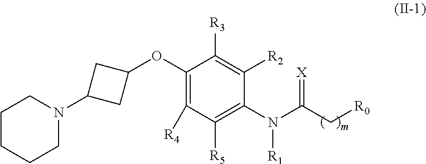
wherein:
X is selected from O or S;
m is selected from 1 or 2;
R₀ is selected from substituted or unsubstituted 5-8 membered heterocyclyl, or substituted or unsubstituted 5-8 membered heteroaryl; wherein the heterocyclyl or heteroaryl is each independently and optionally substituted with one or more substituents each independantly selected from C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆alkylhydroxyl, C₁₋₆ alkylamino, C₁₋₆ alkylamide, C₁₋₆ alkylsulfone, C₁₋₆ alkylsulfoxide, C₁₋₆ haloalkoxy, C₁₋₆ alkoxyhydroxyl, C₁₋₆ alkoxyamino, C₁₋₆alkoxyamide, C₁₋₆ alkoxysulfone, C₁₋₆alkoxysulfoxide, 3-6 membered cycloalkyl, 3-6 membered heterocyclyl, aryl or heteroaryl;
R₁ is selected from hydrogen, C₁₋₆ alkyl, —C(O)-alkyl or —S(O)₂-alkyl;
R₂, R₃, R₄, and R₅ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, or C₁₋₆ alkylsulfoxide;
each of the heterocyclyl, heteroaryl and benzoheterocyclic group contains at least one heteroatom selected from N, O or S.

4. The histamine-H3-receptor antagonist of claim 2, having the structure of formula (II-2) or a pharmaceutically acceptable salt thereof: 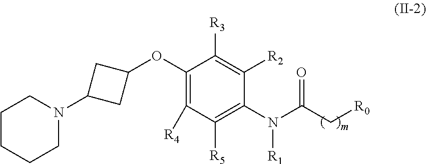
wherein:
m is selected from 1 or 2;
R₀ is selected from —NR′R″ or —O(CH₂)ₘNR′R″;
R′ and R″ are each independently selected from hydrogen, C₁₋₆ alkyl, C₁₋₆ alkoxy, halogen, amino, hydroxy, carboxyl, carbonyl, amide, cyano, C₁₋₆ haloalkyl, C₁₋₆alkylhydroxy, C₁₋₆ alkylamino, C₁₋₆alkylamide, C₁₋₆ haloalkoxy, C₁₋₆alkoxyhydroxy, C₁₋₆alkoxyamino, or C₁₋₆ alkoxyamide, and R′ and R″ are not hydrogen at the same time;
R₁ is selected from hydrogen, C₁₋₆ alkyl, —C(O)-alkyl or —S(O)₂-alkyl;
R₂, R₃, R₄, and R₅ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, or C₁₋₆ alkylsulfoxide.

5. The histamine-H3-receptor antagonist of claim 1, having the structure of formula (III) or a pharmaceutically acceptable salt thereof: 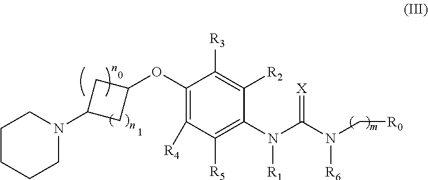
wherein:
X is O or S;
n₀ and n₁ are each independently selected from 1, 2 or 3;
m is selected from 0, 1, 2, 3, 4, 5 or 6;
R₀ is selected from substituted or unsubstituted 5-8 membered heterocyclyl, or substituted or unsubstituted 5-8 membered heteroaryl; wherein the heterocyclyl or heteroaryl is each independently and optionally substituted with one or more substituents each independently selected from C₁₋₆ alkyl, C₁₋₆ alkoxy, C₂₋₆ alkenyl, C₂₋₆ alkynyl, halogen, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, C₁₋₆ alkylsulfoxide, C₁₋₆ haloalkoxy, C₁₋₆ alkoxyhydroxyl, C₁₋₆ alkoxyamino, C₁₋₆ alkoxyamide, C₁₋₆ alkoxysulfone, C₁₋₆ alkoxysulfoxide, 3-6 membered cycloalkyl, 3-6 membered heterocyclyl, aryl or heteroaryl;
R₁ and R₆ are each independently selected from hydrogen, C₁₋₆ alkyl, —C(O)-alkyl or —S(O)₂-alkyl;
R₂, R₃, R₄, R₅ and R₆ are each independently selected from hydrogen, halogen, C₁₋₆ alkyl, C₂₋₆ alkenyl, C₂₋₆ alkynyl, amino, hydroxyl, cyano, amide, sulfone, sulfoxide, C₁₋₆ haloalkyl, C₁₋₆ alkylhydroxyl, C₁₋₆ alkylamino, C₁₋₆ alkylamide, C₁₋₆ alkylsulfone, or C₁₋₆ alkylsulfoxide;
or, R₂, R₃ and the two carbon atoms on the benzene ring to which R₂, R₃ are connected respectively, form a substituted or unsubstituted benzobicyclic structure; wherein the benzobicyclic structure may be a benzoheterocyclic ring, but is not limited thereto;
or, R₄, R₅ and the two carbon atoms on the benzene ring to which R₄, R₅ are connected respectively, form a substituted or unsubstituted benzobicyclic structure; wherein the benzobicyclic structure may be a benzoheterocyclic ring, but is not limited thereto;
each of heterocyclyl, heteroaryl and benzoheterocyclic group contains at least one heteroatom selected from N, O or S.

6. The histamine-H3-receptor antagonist of claim 5, having the structure of formula (III-1) or a pharmaceutically acceptable salt thereof: 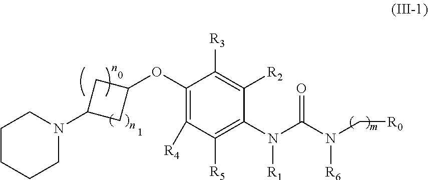
wherein:
n₀ and n₁ are each independently selected from 1, 2 or 3;
m is selected from 0, 1, 2, 3, 4, 5 or 6;
R₀ is selected from substituted or unsubstituted 5-8 membered heterocyclyl, or substituted or unsubstituted 5-8 membered heteroaryl; wherein the heterocyclyl or heteroaryl is each independently and optionally substituted with one or more substituents each independently selected from the group consisting of C₁₋₆ alkyl, C₁₋₆ alkoxy, C₂₋₆ alkenyl, C₂₋₆ alkynyl, halogen, amino, hydroxyl, C₁₋₆ haloalkyl, C₁₋₆ alkylhydroxyl, C₁₋₆ alkylamino, C₁₋₆ haloalkoxy, C₁₋₆ alkoxyhydroxyl, C₁₋₆ alkoxyamino, and 3-6 membered cycloalkyl;
R₂, R₃, R₄, R₅, and R₆ are each independently selected from hydrogen, halogen, C₁₋₆ alkyl, amino, hydroxy, C₁₋₆ haloalkyl, C₁₋₆ alkylhydroxy, or C₁₋₆ alkylamino;
each of the heterocyclyl, heteroaryl and benzoheterocyclic group contains at least one heteroatom selected from N, O or S.

7. A histamine-H3-receptor antagonist having the following structure, or an isomer thereof, or a solvate thereof, or a pharmaceutically acceptable salt thereof:
2-morpholino-N-(4-(3-(piperidin-1-yl)cyclobutoxy)phenyl)acetamide;
2-(2-methylpyrrolidin-1-yl)-N-(4-(3-(piperidin-1-yl)cyclobutoxy)phenyl)acetamide;
2-(piperidin-1-yl)-N-(4-(3-(piperidin-1-yl)cyclobutoxy)phenyl)acetamide;
2-(4-methyl-1,4-diazepan-1-yl)-N-(4-(3-(piperidin-1-yl)cyclobutoxy)phenyl)acetamide;
2-(4,4-difluoropiperidin-1-yl)-N-(4-(3-(piperidin-1-yl)cyclobutoxy)phenyl)acetamide;
2-(4-hydroxypiperidin-1-yl)-N-(4-(3-(piperidin-1-yl)cyclobutoxy)phenyl)acetamide;
N-(4-(3-(piperidin-1-yl)cyclobutoxy)phenyl)-2-thiomorpholinoacetamide;
2-(3-(2-hydroxyethyl)piperidin-1-yl)-N-(4-(3-(piperidin-1-yl)cyclobutoxy)phenyl)acetamide;
2-(piperazin-1-yl)-N-(4-(3-(piperidin-1-yl)cyclobutoxy)phenyl)acetamide;
2-(4-methylpiperazin-1-yl)-N-(4-(3-(piperidin-1-yl)cyclobutoxy)phenyl)acetamide;
2-(3-hydroxypiperidin-1-yl)-N-(4-(3-(piperidin-1-yl)cyclobutoxy)phenyl)acetamide;
2-(4-cyclobutylpiperazin-1-yl)-N-(4-(3-(piperidin-1-yl)cyclobutoxy)phenyl)acetamide;
1-(2-morpholinoethyl)-3-(4-(3-(piperidin-1-yl)cyclobutoxy)phenyl)urea;
1-(2-(2-methylpyrrolidin-1-yl)ethyl)-3-(4-(3-(piperidin-1-yl)cyclobutoxy)phenyl)urea;
1-(4-(3-(piperidin-1-yl)cyclobutoxy)phenyl)-3-(2-(piperidin-1-yl)ethyl)urea;
1-(2-(piperazin-1-yl)ethyl)-3-(4-(3-(piperidin-1-yl)cyclobutoxy)phenyl)urea;
1-(2-(4-methyl-1,4-diazepan-1-yl)ethyl)-3-(4-(3-(piperidin-1-yl)cyclobutoxy)phenyl)urea;
1-(2-(4,4-difluoropiperidin-1-yl)ethyl)-3-(4-(3-(piperidin-1-yl)cyclobutoxy)phenyl)urea;
1-(2-(4-hydroxypiperidin-1-yl)ethyl)-3-(4-(3-(piperidin-1-yl)cyclobutoxy)phenyl)urea;
1-(4-(3-(piperidin-1-yl)cyclobutoxy)phenyl)-3-(2-thiomorpholinoethyl)urea;
1-(2-(2-(2-hydroxyethyl)piperidin-1-yl)ethyl)-3-(4-(3-(piperidin-1-yl)cyclobutoxy)phenyl)urea;
1-(2-(4-methylpiperazin-1-yl)ethyl)-3-(4-(3-(piperidin-1-yl)cyclobutoxy)phenyl)urea;
1-(2-(3-hydroxypiperidin-1-yl)ethyl)-3-(4-(3-(piperidin-1-yl)cyclobutoxy)phenyl)urea;
1-(2-(4-cyclobutylpiperazin-1-yl)ethyl)-3-(4-(3-(piperidin-1-yl)cyclobutoxy)phenyl)urea;
2-morpholino-N-(4-(3-(piperidin-1-yl)cyclobutoxy)phenyl)ethanethioamide;
2-(2-methylpyrrolidin-1-yl)-N-(4-(3-(piperidin-1-yl)cyclobutoxy)phenyl)ethanethioamide;
2-(piperidin-1-yl)-N-(4-(3-(piperidin-1-yl)cyclobutoxy)phenyl)ethanethioamide;
2-(4-methyl-1,4-diazepan-1-yl)-N-(4-(3-(piperidin-1-yl)cyclobutoxy)phenyl)ethanethioamide;
2-(diethylamino)-N-(4-(3-(piperidin-1-yl)cyclobutoxy)phenyl)acetamide;
2-(dimethylamino)-N-(4-(3-(piperidin-1-yl)cyclobutoxy)phenyl)acetamide; or
2-(2-(dimethylamino)ethoxy)-N-(4-(3-(piperidin-1-yl)cyclobutoxy)phenyl)acetamide.

8. A histamine-H3-receptor antagonist having the following structure or a pharmaceutically acceptable salt thereof:
(R)-2-(2-methylpyrrolidin-1-yl)-N-(4-(3-(piperidin-1-yl)cyclobutoxy)phenyl)acetamide;
(R)-1-(2-(2-methylpyrrolidin-1-yl)ethyl)-3-(4-(3-(piperidin-1-yl)cyclobutoxy)phenyl)urea; or
(R)-2-(2-methylpyrrolidin-1-yl)-N-(4-(3-(piperidin-1-yl)cyclobutoxy)phenyl)ethanethioamide.

9. A pharmaceutical composition comprising an antagonist of claim 1 or a pharmaceutically acceptable salt thereof, and at least one pharmaceutically acceptable carrier or excipient.

10. A method for preventing or treating a disease or disorder associated with histamine H3 receptor in a subject, comprising administration of a therapeutically effective amount of an antagonist of claim 1 or a pharmaceutically acceptable salt thereof, to the subject in need of.

11. The method of claim 10, wherein the disease or disorder associated with histamine H3 receptor is cognitive impairment, dementia, attention deficit hyperactivity disorder, schizophrenia, epilepsy, sleep disorders, sleep apnea, obesity, eating disorders, pain or itching.

12. The method of claim 10, wherein the disease or disorder associated with histamine H3 receptor is neuropathic pain.

13. The method of claim 12, wherein the neuropathic pain is peripheral neuropathic pain or central neuropathic pain.
